(19) United States
(12) Patent Application Publication
(10) Pub. No.: US 2024/0352536 A1
(43) Pub. Date: Oct. 24, 2024

(54) Assay for Predicting Risk of Capecitabine-Induced Toxicity in a Subject

(71) Applicants: Oxford University Innovation Ltd., Oxford, Oxfordshire (GB); The University of Birmingham, Edgbaston, Birmingham (GB)

(72) Inventors: Claire PALLES, Birmingham (GB); Ik SHIN CHIN, Birmingham (GB)

(21) Appl. No.: 18/687,177

(22) PCT Filed: Aug. 26, 2022
(86) PCT No.: PCT/GB2022/052195

§ 371 Date: Feb. 27, 2024

(30) Foreign Application Priority Data
Aug. 27, 2021 (GB) 2112296.5

(51) Int. Cl. C12Q 1/6886

(52) U.S. Cl. CPC C12Q 1/6886; C12Q 2600/118; C12Q 2600/156

(57) ABSTRACT

The invention provides an assay useful in predicting risk of capecitabine-induced toxicity in a subject. The subject may be screened for the presence of at least one polymorphism of the invention. Suitable polymorphisms are provided. The presence of one or more of the polymorphisms indicates an increased risk of developing capecitabine-induced toxicity; a negative result may indicate a decreased risk of developing capecitabine-induced toxicity.

The invention relates to an assay. More specifically, the invention relates to an assay useful in predicting toxicity of a chemotherapeutic agent. Reagents and kits for carrying out the assay are described.

Capecitabine (pentyl [1-(3,4-dihydroxy-5-methyltetrahydrofuran-2-yl)-5-fluoro-2-oxo-1H-pyrimidin-4-yl]carbamate) is an oral prodrug of 5-fluorouracil (5-FU) chemotherapy that is widely used in many cancer types, especially in gastrointestinal, breast cancer and colorectal cancer. Chemotherapy regimens often involve administration of capecitabine as a course of several ‘cycles’ of treatment. Typically, a treatment cycle lasts over three weeks, although this is tailored according to the patient and the cancer type. For example, a three-week cycle may include daily administration of capecitabine for a period of two weeks, followed by a week with no administration of capecitabine for a week.

Capecitabine causes cytotoxicity by inhibiting production of thymidine and by being converted to metabolites that are incorporated into DNA and RNA (Noordhuis P et al., Nucleosides Nucleotides & Nucleic Acids 23: 1481-1484 (2004), incorporated herein by reference). As with other 5-FU-based chemotherapy regimens, approximately one third of capecitabine patients suffer dose-limiting levels of drug-induced adverse events. Not only are the associated toxicities common, they are potentially fatal. 10-30% of patients administered 5-FU-based therapy suffer substantial toxicities, defined as Grade 3 or above as measured using the NCI Common Toxicity Criteria for Adverse Events (CTCAE) version 3.0). Approximately a third of patients with colorectal cancer experienced serious (CTCAE≥grade 3) toxicities from 5-FU with a fatality rate of approximately 1% (Meulendijks D et al., Cancer Treat Rev (2016) Nov. 1; 50:23-34).

NCI Common Toxicity Criteria for Adverse Events (CTCAE) version 3.0):

CTCAE v. 3.0

| GRADE | MEANING |
|---|---|
| 1 | Mild Adverse Event |
| 2 | Moderate Adverse Event |
| 3 | Severe Adverse Event |
| 4 | Life-threatening or disabling Adverse Event |
| 5 | Death related to Adverse Event |

Since capecitabine is an oral prodrug of 5-FU, the terms “5-FU-induced toxicities”, “5-FU toxicities”, “capecitabine-induced toxicities”, “capecitabine toxicities” (and the like) may be used interchangeably.

Capecitabine-induced toxicities typically include symptoms such as diarrhoea, nausea and vomiting, mucositis/stomatitis, myelosuppression, neutropaenia, thrombocytopaenia, and hand-foot syndrome (HFS). The most common dose-limiting capecitabine-induced toxicities are HFS and diarrhoea. There is wide variation in the frequency and type of toxicity depending upon the schedule of administration, and overall there is a 0.5-1.0% mortality (Grade 5 as measured using CTCAE version 3.0) associated with 5-FU use (Grem J L., Investigational new drugs 18(4): 299-313 (2000); and Twelves C et al., The New England journal of medicine 352(26): 2696-704 (2005), both incorporated herein by reference). The onset of toxicity may be rapid, which results in mortality for 0.5% to 2% of patients in monotherapy and combination regimens of infusional and bolus 5-FU (Saltz L B et al., J Clin Oncol. 25(23): 3456-3461 (2007) incorporated herein by reference), and about half that number for capecitabine schedules. Inter-patient differences in toxicity may be explained by clinical factors such as patient age, gender, local clinical practice and diet (Stein B N et al., Cancer 75(1): 11-17 (1995); Cassidy J et al., Ann Oncol. 13(4): 566-575 (2002); Haller D G et al., J Clin Oncol. 26(13): 2118-2123 (2008), each incorporated herein by reference). However, much variability in toxicity remains unexplained.

Consequently, much attention has focused on the identification of biomarkers or assays predictive of 5-FU toxicity (Boisdron-Celle M et al., Cancer letters 249(2): 271-82 (2007); and Saif M W et al., Journal of the National Cancer Institute 101(22): 1543-52 (2009), both incorporated herein by reference). However, 5-FU metabolism is complex, with multiple enzymatic reactions and intermediates.

The biochemical pathway of capecitabine activation and subsequent 5-FU action and degradation is well-established and provides 25 candidate genes in which variation might affect 5-FU toxicity (FIG. 1) (Longley D B et al., Nat Rev Cancer 3(5): 330-338 (2003); Thorn C F et al., Pharmacogenet Genomics 21(4): 237-242_(2011); West C M et al., Nat Rev Cancer 4(6): 457-469 (2004); Miwa M et al., Eur J Cancer 34(8): 1274-1281 (1998), each incorporated herein by reference). Upon absorption in the gut, capecitabine is partially converted to 5-FU in the liver, then preferentially converted to 5-FU at the tumour site. Much 5-FU (˜80%) is degraded in the liver by dihydropyrimidine dehydrogenase (DPYD) prior to activation. As part of the drug's rationally-designed activation, 5-FU is further activated in the tumour to cytotoxic compounds that inhibit DNA synthesis by competing with nucleotide precursors for binding with thymidylate synthase (TYMS). Various sources of toxicity may exist, including alternative activation pathways outside the tumour that result in direct DNA/RNA damage through incorporation, undesired transport of activated compounds, variable expression of drug targets, and reduced levels of drug degradation.

As noted above, the majority of 5-FU is catabolised and inactivated by dihydropyrimidine dehydrogenase (DPD), which is the rate-limiting step of the drug's metabolism pathway (Diasio R B, et al. Clin Pharmacokinet. (1989); 16(4):215-37). Four variants mapping to the DPYD gene which encodes DPD have been identified by the Clinical Pharmacogenomics Implementation Consortium (CPIC) as having sufficient evidence to be used in clinical practice to screen for complete or partial DPD deficiency to guide subsequent 5-FU chemotherapy dose modification (Amstutz U, et al: Clin Pharmacol Ther. (2018) February; 103(2):210-6). However, the current strategy of testing for these four variants has low sensitivity, probably owing to failure to test for other toxicity associated variants mapping to within and outside the fluoropyrimidine metabolism pathway.

Capecitabine is activated to 5-FU via a three-step enzymatic process via carboxylesterase (CES), cytidine deaminase (CDA) and thymidine phosphorylase (TYMP) (Lam S W, et al. Cancer Treat Rev (2016); 50:9-22). Several pharmacogenetic studies have reported capecitabine toxicity associated variants in 5-FU metabolising enzymes thymidylate synthase (TYMS) and DPYD, capecitabine activating enzymes CDA and CES2 and methylenetetrahydrofolate reductase (MTHFR) (Lam S W, et al. Cancer Treat Rev (2016); 50:9-22). However, the reported findings so far have been inconsistent between studies possibly because of small sample sizes, heterogeneity in sample populations and different drug regimens (Lam S W, et al. Cancer Treat Rev (2016); 50:9-22).

Commercially available kits aimed at identifying capecitabine-induced toxicity risk are not optimal and tend to identify common polymorphisms, thus classifying nearly every test subject as being at risk. Even kits which do not include such common polymorphisms typically provide no better than 29% sensitivity.

There is therefore currently no reliable way of predicting adverse events, since it is unclear which (if any) genetic variants make good predictors of capecitabine-induced toxicity.

There is accordingly a need for certainty regarding which genetic variants are truly predictive of adverse events from capecitabine. There is a need for clinical biomarkers and assays predictive of capecitabine-induced toxicity.

The invention addresses one or more of the above needs. In particular, the inventors have discovered clinical biomarkers and related assays that are predictive of capecitabine-induced toxicity.

Accordingly, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof.

In one embodiment, the invention provides a method of screening for risk of capecitabine-induced toxicity in a subject, comprising screening the subject for the presence of at least one polymorphism selected from: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the capecitabine-induced toxicity is early onset capecitabine-induced toxicity. “Early onset” capecitabine-induced toxicity occurs during cycle 1 and/or cycle 2 of capecitabine chemotherapy.

Functionally equivalent variants of the polymorphisms may also be screened for.

Screening for risk of capecitabine-induced toxicity comprises screening for the presence or absence of alleles which are associated with toxicity risk (referred to herein as “toxicity risk alleles”). Toxicity risk alleles are provided in Tables 2 and 4. It will therefore be understood that “polymorphisms of the invention” (and the like) are alleles of the polymorphisms that are associated with toxicity risk.

A positive result (i.e. the presence of one or more toxicity risk alleles of polymorphisms of the invention) indicates an increased risk of developing capecitabine-induced toxicity. A negative result (i.e. absence of any of the toxicity risk alleles at screened-for polymorphism(s) of the invention) may indicate no increased risk or a decreased risk of developing capecitabine-induced toxicity.

Each of rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836 are independently associated with either early onset (cycle 1 or cycle 2) capecitabine-induced grade 3 toxicities and/or ≥grade 3 toxicity events experienced across all treatment cycles at genome-wide significance.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945; and rs73039836, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945; and rs73039836, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945; and rs73039836, and functionally equivalent variants thereof;

wherein a negative result indicates a decreased risk of developing capecitabine-induced toxicity compared to a subject which possesses said at least one polymorphism. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof;

wherein a negative result indicates a decreased risk of developing capecitabine-induced toxicity compared to a subject which possesses said at least one polymorphism. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof;

wherein a negative result indicates a decreased risk of developing capecitabine-induced toxicity compared to a subject which possesses said at least one polymorphism. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945; and rs73039836, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is an early onset (cycle 1 or cycle 2) capecitabine-induced ≥grade 3 toxicity and/or a ≥grade 3 toxicity event experienced across all treatment cycles.

In one embodiment, the method comprises screening the subject for 2 or more (e.g. 2, 3, 4, 5, 6, 7, 8, 9, 10, 11, 12, 13, 14, 15, 16, 17, 18, 19, 20, 21, 22, 23, 24, 25, 30, 35, 40, 45, or 50) of rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945; and rs73039836.

In one embodiment, the method comprises screening the subject for 2 or more (e.g. 2, 3, 4, 5, 6, 7, 8, 9, 10, 11, 12, 13, 14, 15, 16, 17, 18, 19, 20, 21, 22, 23, 24 or 25) of rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836.

In one embodiment, the method comprises screening the subject for 2 or more (e.g. 2, 3, 4, 5, 6, 7, 8, 9, 10, 11, 12, 13, 14, 15, 16, 17, 18, 19, 20, 21, 22, 23, 24, 25, or 26) of rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887.

The Inventors have found that screening for rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887 provides up to 64.79% sensitivity, 69.5% specificity and AUC of 72.8% as a measure of the performance of the classifier for early onset capecitabine-induced ≥grade 3 toxicity and up to 47.73% sensitivity, 75.85% specificity and AUC of 69.3% as a measure of the performance of the classifier for any cycle capecitabine-induced grade 3+ toxicity.

Each of rs147827021; rs149994224; rs111378975; and rs76211390 are independently associated with both early onset and all cycle ≥grade 3 events.

In one embodiment, the invention provides a method of screening for risk of capecitabine-induced toxicity in a subject, comprising screening the subject for the presence of at least one polymorphism selected from: rs147827021; rs149994224; rs111378975; and rs76211390, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is a capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs147827021; rs149994224; rs111378975; and rs76211390, and functionally equivalent variants thereof;

wherein a negative result indicates a decreased risk of developing capecitabine-induced toxicity compared to a subject which possesses said at least one polymorphism. In one embodiment, the capecitabine-induced toxicity is a capecitabine-induced ≥grade 3 toxicity and/or a ≥grade 3 toxicity event experienced across all treatment cycles.

In one embodiment, the invention provides a method of screening for risk of capecitabine-induced toxicity in a subject, comprising screening the subject for the presence of at least one polymorphism selected from: rs147827021; rs149994224; rs111378975; and rs76211390, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is a capecitabine-induced ≥grade 3 toxicity and/or a ≥grade 3 toxicity event experienced across all treatment cycles.

In one embodiment, the method comprises screening the subject for 2 or more (e.g. 2, 3, or 4) of rs147827021; rs149994224; rs111378975; and rs76211390. In one embodiment, the method comprises screening the subject for each of rs147827021; rs149994224; and rs111378975. In one embodiment, the method comprises screening the subject for rs76211390.

Each of rs76211390 and rs79272399 are independently associated with early onset grade 3+ mucositis toxicity.

In one embodiment, the invention provides a method of screening for risk of capecitabine-induced toxicity in a subject, comprising screening the subject for the presence of at least one polymorphism selected from: rs76211390 and rs79272399, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset grade 3+mucositis.

In one embodiment, the invention provides a method of screening for risk of capecitabine-induced toxicity in a subject, comprising screening the subject for the presence of at least one polymorphism selected from: rs76211390 and rs79272399, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset grade 3+mucositis.

In one embodiment, the invention provides a method of screening for risk of capecitabine-induced toxicity in a subject, comprising screening the subject for the presence of at least one polymorphism selected from: rs76211390 and rs79272399, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset grade 3+mucositis.

In one embodiment, the method comprises screening the subject for each of rs76211390 and rs79272399.

Each of rs76211390; rs77911328; rs145225093; and rs117241924 are independently associated with ≥grade 3 mucositis experienced across all treatment cycles.

In one embodiment, the invention provides a method of screening for risk of capecitabine-induced toxicity in a subject, comprising screening the subject for the presence of at least one polymorphism selected from: rs76211390; rs77911328; rs145225093; and rs117241924, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is grade 3 mucositis experienced across all treatment cycles.

In one embodiment, the invention provides a method of screening for risk of capecitabine-induced toxicity in a subject, comprising screening the subject for the presence of at least one polymorphism selected from: rs76211390; rs77911328; rs145225093; and rs117241924, and functionally equivalent variants thereof;

wherein a negative result indicates a decreased risk of developing capecitabine-induced toxicity compared to a subject which possesses said at least one polymorphism. In one embodiment, the capecitabine-induced toxicity is grade 3 mucositis experienced across all treatment cycles.

In one embodiment, the invention provides a method of screening for risk of capecitabine-induced toxicity in a subject, comprising screening the subject for the presence of at least one polymorphism selected from: rs76211390; rs77911328; rs145225093; and rs117241924, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is grade 3 mucositis experienced across all treatment cycles.

In one embodiment, the method comprises screening the subject for 2 or more (e.g. 2, 3, or 4) of rs76211390; rs77911328; rs145225093; and rs117241924. In one embodiment, the method comprises screening the subject for each of: rs76211390; rs77911328; rs145225093; and rs117241924.

Each of rs76211390; rs79272399; rs77911328; rs145225093; and rs117241924 are independently associated with capecitabine-induced mucositis toxicity.

In one embodiment, the invention provides a method of screening for risk of capecitabine-induced toxicity in a subject, comprising screening the subject for the presence of at least one polymorphism selected from: rs76211390; rs79272399; rs77911328; rs145225093; and rs117241924, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is mucositis toxicity.

In one embodiment, the invention provides a method of screening for risk of capecitabine-induced toxicity in a subject, comprising screening the subject for the presence of at least one polymorphism selected from: rs76211390; rs79272399; rs77911328; rs145225093; and rs117241924, and functionally equivalent variants thereof;

wherein a negative result indicates a decreased risk of developing capecitabine-induced toxicity compared to a subject which possesses said at least one polymorphism. In one embodiment, the capecitabine-induced toxicity is mucositis toxicity.

In one embodiment, the invention provides a method of screening for risk of capecitabine-induced toxicity in a subject, comprising screening the subject for the presence of at least one polymorphism selected from: rs76211390; rs79272399; rs77911328; rs145225093; and rs117241924, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is mucositis toxicity.

In one embodiment, the method comprises screening the subject for 2 or more (e.g. 2, 3, 4, or 5) of rs76211390; rs79272399; rs77911328; rs145225093; and rs117241924. In one embodiment, the method comprises screening the subject for each of: rs76211390, rs79272399, rs77911328; rs145225093 and rs117241924.

Screening for rs76211390; rs79272399; rs77911328; rs145225093; and rs117241924 has surprisingly been found to provide up to 100% sensitivity, 91.50% specificity and AUC of 98.7% as a measure of the performance of the classifier for early onset grade 3+mucositis toxicity. rs76211390, rs79272399, rs77911328; rs145225093 and rs117241924 also provide up to 100% sensitivity, 85.65% specificity and area under the curve (AUC) of 98.0% as a measure of the performance of the classifier for ≥grade 3 mucositis experienced across all treatment cycles.

Each of rs76211390; rs79272399; rs2929329; rs35741121; rs56329496; rs117810108; rs149994224; rs11024884; and rs111378975 are independently associated with early onset grade 3+mucositis toxicity.

In one embodiment, the invention provides a method of screening for risk of capecitabine-induced toxicity in a subject, comprising screening the subject for the presence of at least one polymorphism selected from: rs76211390; rs79272399; rs2929329; rs35741121; rs56329496; rs117810108; rs149994224; rs11024884; and rs111378975, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset grade 3+mucositis.

In one embodiment, the invention provides a method of screening for risk of capecitabine-induced toxicity in a subject, comprising screening the subject for the presence of at least one polymorphism selected from: rs76211390; rs79272399; rs2929329; rs35741121; rs56329496; rs117810108; rs149994224; rs11024884; and rs111378975, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset grade 3+mucositis.

In one embodiment, the invention provides a method of screening for risk of capecitabine-induced toxicity in a subject, comprising screening the subject for the presence of at least one polymorphism selected from: rs76211390; rs79272399; rs2929329; rs35741121; rs56329496; rs117810108; rs149994224; rs11024884; and rs111378975, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset grade 3+mucositis.

In one embodiment, the method comprises screening the subject for 2 or more (e.g. 2, 3, 4, 5, 6, 7, 8, or 9) of rs76211390; rs79272399; rs2929329; rs35741121; rs56329496; rs117810108; rs149994224; rs11024884; and rs111378975.

Each of rs76211390; rs77911328; rs145225093; rs117241924; rs2420201; rs11375020; rs75504333; rs11447080; rs74380254; rs66842063; rs149994224; rs111378975; rs180742844; and rs527945 are independently associated with any cycle mucositis grade 3 toxicity.

In one embodiment, the invention provides a method of screening for risk of capecitabine-induced toxicity in a subject, comprising screening the subject for the presence of at least one polymorphism selected from: rs76211390; rs77911328; rs145225093; rs117241924; rs2420201; rs11375020; rs75504333; rs11447080; rs74380254; rs66842063; rs149994224; rs111378975; rs180742844; and rs527945, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is grade 3 mucositis experienced across all treatment cycles.

In one embodiment, the invention provides a method of screening for risk of capecitabine-induced toxicity in a subject, comprising screening the subject for the presence of at least one polymorphism selected from: rs76211390; rs77911328; rs145225093; rs117241924; rs2420201; rs11375020; rs75504333; rs11447080; rs74380254; rs66842063; rs149994224; rs111378975; rs180742844; and rs527945, and functionally equivalent variants thereof;

wherein a negative result indicates a decreased risk of developing capecitabine-induced toxicity compared to a subject which possesses said at least one polymorphism. In one embodiment, the capecitabine-induced toxicity is grade 3 mucositis experienced across all treatment cycles.

In one embodiment, the invention provides a method of screening for risk of capecitabine-induced toxicity in a subject, comprising screening the subject for the presence of at least one polymorphism selected from: rs76211390; rs77911328; rs145225093; rs117241924; rs2420201; rs11375020; rs75504333; rs11447080; rs74380254; rs66842063; rs149994224; rs111378975; rs180742844; and rs527945, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is grade 3 mucositis experienced across all treatment cycles.

In one embodiment, the method comprises screening the subject for 2 or more (e.g. 2, 3, 4, 5, 6, 7, 8, 9, 10, 11, 12, 13, or 14) of rs76211390; rs77911328; rs145225093; rs117241924; rs2420201; rs11375020; rs75504333; rs11447080; rs74380254; rs66842063; rs149994224; rs111378975; rs180742844; and rs527945.

Each of rs76211390; rs79272399; rs77911328; rs145225093; rs117241924; rs2929329; rs35741121; rs56329496; rs117810108; rs149994224; rs11024884; rs111378975, rs2420201; rs11375020; rs75504333; rs11447080; rs74380254; rs66842063; rs180742844; and rs527945 are independently associated with capecitabine-induced mucositis toxicity.

In one embodiment, the invention provides a method of screening for risk of capecitabine-induced toxicity in a subject, comprising screening the subject for the presence of at least one polymorphism selected from: rs76211390; rs79272399; rs77911328; rs145225093; rs117241924; rs2929329; rs35741121; rs56329496; rs117810108; rs149994224; rs11024884; rs111378975, rs2420201; rs11375020; rs75504333; rs11447080; rs74380254; rs66842063; rs180742844; and rs527945, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is mucositis toxicity.

In one embodiment, the invention provides a method of screening for risk of capecitabine-induced toxicity in a subject, comprising screening the subject for the presence of at least one polymorphism selected from: rs76211390; rs79272399; rs77911328; rs145225093; rs117241924; rs2929329; rs35741121; rs56329496; rs117810108; rs149994224; rs11024884; rs111378975, rs2420201; rs11375020; rs75504333; rs11447080; rs74380254; rs66842063; rs180742844; and rs527945, and functionally equivalent variants thereof;

wherein a negative result indicates a decreased risk of developing capecitabine-induced toxicity compared to a subject which possesses said at least one polymorphism. In one embodiment, the capecitabine-induced toxicity is mucositis toxicity.

In one embodiment, the invention provides a method of screening for risk of capecitabine-induced toxicity in a subject, comprising screening the subject for the presence of at least one polymorphism selected from: rs76211390; rs79272399; rs77911328; rs145225093; rs117241924; rs2929329; rs35741121; rs56329496; rs117810108; rs149994224; rs11024884; rs111378975, rs2420201; rs11375020; rs75504333; rs11447080; rs74380254; rs66842063; rs180742844; and rs527945, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is mucositis toxicity.

In one embodiment, the method comprises screening the subject for 2 or more (e.g. 2, 3, 4, 5, 6, 7, 8, 9, 10, 11, 12, 13, 14, 15, 16, 17, 18, 19, or 20) of rs76211390; rs79272399; rs77911328; rs145225093; rs117241924; rs2929329; rs35741121; rs56329496; rs117810108; rs149994224; rs11024884; rs111378975, rs2420201; rs11375020; rs75504333; rs11447080; rs74380254; rs66842063; rs180742844; and rs527945.

Each of rs2929329; rs35741121; rs56329496; rs117810108; rs149994224; rs11024884; and rs111378975 are independently associated with early onset grade 3+mucositis toxicity.

In one embodiment, the invention provides a method of screening for risk of capecitabine-induced toxicity in a subject, comprising screening the subject for the presence of at least one polymorphism selected from: rs2929329; rs35741121; rs56329496; rs117810108; rs149994224; rs11024884; and rs111378975, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset grade 3+mucositis.

In one embodiment, the invention provides a method of screening for risk of capecitabine-induced toxicity in a subject, comprising screening the subject for the presence of at least one polymorphism selected from: rs2929329; rs35741121; rs56329496; rs117810108; rs149994224; rs11024884; and rs111378975, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset grade 3+mucositis.

In one embodiment, the invention provides a method of screening for risk of capecitabine-induced toxicity in a subject, comprising screening the subject for the presence of at least one polymorphism selected from: rs2929329; rs35741121; rs56329496; rs117810108; rs149994224; rs11024884; and rs111378975, and functionally equivalent variants thereof; wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset grade 3+mucositis.

Screening for rs2929329; rs35741121; rs56329496; rs117810108; rs149994224; rs11024884; and rs111378975 has surprisingly been found to provide up to 100% sensitivity, 90.20% specificity and area under the curve (AUC) of 98.7% as a measure of the performance of the classifier for early onset grade 3+mucositis toxicity.

In one embodiment, the method comprises screening the subject for 2 or more (e.g. 2, 3, 4, 5, 6 or 7) of rs2929329; rs35741121; rs56329496; rs117810108; rs149994224; rs11024884; and rs111378975. In one embodiment, the method comprises screening the subject for each of: rs2929329; rs35741121; rs56329496; rs117810108; rs149994224; rs11024884; and rs111378975.

Each of rs2420201; rs11375020; rs75504333; rs11447080; rs74380254; rs66842063; rs149994224; rs111378975; rs180742844; and rs527945 are independently associated with any cycle mucositis grade 3+toxicity.

In one embodiment, the invention provides a method of screening for risk of capecitabine-induced toxicity in a subject, comprising screening the subject for the presence of at least one polymorphism selected from: rs2420201; rs11375020; rs75504333; rs11447080; rs74380254; rs66842063; rs149994224; rs111378975; rs180742844; and rs527945, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle grade 3+mucositis.

In one embodiment, the invention provides a method of screening for risk of capecitabine-induced toxicity in a subject, comprising screening the subject for the presence of at least one polymorphism selected from: rs2420201; rs11375020; rs75504333; rs11447080; rs74380254; rs66842063; rs149994224; rs111378975; rs180742844; and rs527945, and functionally equivalent variants thereof;
wherein a negative result indicates a decreased risk of developing capecitabine-induced toxicity compared to a subject which possesses said at least one polymorphism. In one embodiment, the capecitabine-induced toxicity is any cycle grade 3+mucositis.

In one embodiment, the invention provides a method of screening for risk of capecitabine-induced toxicity in a subject, comprising screening the subject for the presence of at least one polymorphism selected from: rs2420201; rs11375020; rs75504333; rs11447080; rs74380254; rs66842063; rs149994224; rs111378975; rs180742844; and rs527945, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle grade 3+mucositis.

Screening for rs2420201; rs11375020; rs75504333; rs11447080; rs74380254; rs66842063; rs149994224; rs111378975; rs180742844; and rs527945 has surprisingly been found to provide up to 100% sensitivity, 98.36% specificity and AUC of 99.7% as a measure of the performance of the classifier for any cycle mucositis grade 3+toxicity.

In one embodiment, the method comprises screening the subject for 2 or more (e.g. 2, 3, 4, 5, 6, 7, 8, 9 or 10) of rs2420201; rs11375020; rs75504333; rs11447080; rs74380254; rs66842063; rs149994224; rs111378975; rs180742844; and rs527945. In one embodiment, the method comprises screening the subject for each of: rs2420201; rs11375020; rs75504333; rs11447080; rs74380254; rs66842063; rs149994224; rs111378975; rs180742844; and rs527945.

Each of rs2929329; rs35741121; rs56329496; rs117810108; rs149994224; rs11024884; rs111378975, rs2420201; rs11375020; rs75504333; rs11447080; rs74380254; rs66842063; rs180742844; and rs527945 are independently associated with mucositis toxicity.

In one embodiment, the invention provides a method of screening for risk of capecitabine-induced toxicity in a subject, comprising screening the subject for the presence of at least one polymorphism selected from: rs2929329; rs35741121; rs56329496; rs117810108; rs149994224; rs11024884; rs111378975, rs2420201; rs11375020; rs75504333; rs11447080; rs74380254; rs66842063; rs180742844; and rs527945, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is mucositis toxicity.

In one embodiment, the invention provides a method of screening for risk of capecitabine-induced toxicity in a subject, comprising screening the subject for the presence of at least one polymorphism selected from: rs2929329; rs35741121; rs56329496; rs117810108; rs149994224; rs11024884; rs111378975, rs2420201; rs11375020; rs75504333; rs11447080; rs74380254; rs66842063; rs180742844; and rs527945, and functionally equivalent variants thereof;

wherein a negative result indicates a decreased risk of developing capecitabine-induced toxicity compared to a subject which possesses said at least one polymorphism. In one embodiment, the capecitabine-induced toxicity is mucositis toxicity.

In one embodiment, the invention provides a method of screening for risk of capecitabine-induced toxicity in a subject, comprising screening the subject for the presence of at least one polymorphism selected from: rs2929329; rs35741121; rs56329496; rs117810108; rs149994224; rs11024884; rs111378975, rs2420201; rs11375020; rs75504333; rs11447080; rs74380254; rs66842063; rs180742844; and rs527945, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is mucositis toxicity.

In one embodiment, the method comprises screening the subject for 2 or more (e.g. 2, 3, 4, 5, 6, 7, 8, 9, 10, 11, 12, 13, 14 or 15) of rs2929329; rs35741121; rs56329496; rs117810108; rs149994224; rs11024884; rs111378975, rs2420201; rs11375020; rs75504333; rs11447080; rs74380254; rs66842063; rs180742844; and rs527945.

Each of rs75217739 and rs139788123 are independently associated with any cycle haematological toxicity.

In one embodiment, the invention provides a method of screening for risk of capecitabine-induced toxicity in a subject, comprising screening the subject for the presence of at least one polymorphism selected from: rs75217739 and rs139788123, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle haematological toxicity.

In one embodiment, the invention provides a method of screening for risk of capecitabine-induced toxicity in a subject, comprising screening the subject for the presence of at least one polymorphism selected from: rs75217739 and rs139788123, and functionally equivalent variants thereof;

wherein a negative result indicates a decreased risk of developing capecitabine-induced toxicity compared to a subject which possesses said at least one polymorphism. In one embodiment, the capecitabine-induced toxicity is any cycle haematological toxicity.

In one embodiment, the invention provides a method of screening for risk of capecitabine-induced toxicity in a subject, comprising screening the subject for the presence of at least one polymorphism selected from: rs75217739 and rs139788123, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle haematological toxicity.

Screening for rs75217739 and rs139788123 has been found to provide up to 75% sensitivity, 89.62% specificity and AUC of 80.0% as a measure of the performance of the classifier for early onset haematological toxicity and 68.42% sensitivity, 86.56% specificity and AUC of 74.4% as a measure of the performance of the classifier for grade 3+ haematological toxicity at any cycle.

In one embodiment, the method comprises screening the subject for both of rs75217739 and rs139788123.

Each of rs75217739; rs139788123; rs147827021 and rs6137844 are independently associated with any cycle haematological toxicity.

In one embodiment, the invention provides a method of screening for risk of capecitabine-induced toxicity in a subject, comprising screening the subject for the presence of at least one polymorphism selected from: rs75217739; rs139788123; rs147827021 and rs6137844, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle haematological toxicity.

In one embodiment, the invention provides a method of screening for risk of capecitabine-induced toxicity in a subject, comprising screening the subject for the presence of at least one polymorphism selected from: rs75217739; rs139788123; rs147827021 and rs6137844, and functionally equivalent variants thereof;

wherein a negative result indicates a decreased risk of developing capecitabine-induced toxicity compared to a subject which possesses said at least one polymorphism. In one embodiment, the capecitabine-induced toxicity is any cycle haematological toxicity.

In one embodiment, the invention provides a method of screening for risk of capecitabine-induced toxicity in a subject, comprising screening the subject for the presence of at least one polymorphism selected from: rs75217739; rs139788123; rs147827021 and rs6137844, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle haematological toxicity.

In one embodiment, the method comprises screening the subject for 2 or more (e.g. 2, 3, or 4) of rs75217739; rs139788123; rs147827021 and rs6137844. In one embodiment, the method comprises screening the subject for each of rs75217739; rs139788123; rs147827021 and rs6137844.

Each of rs75217739; rs139788123; rs698607; rs115143613; rs147827021; rs199983532; rs1327306; rs4792825; rs78361592; and rs6137844 are independently associated with haematological toxicity.

In one embodiment, the invention provides a method of screening for risk of capecitabine-induced toxicity in a subject, comprising screening the subject for the presence of at least one polymorphism selected from: rs75217739; rs139788123; rs698607; rs115143613; rs147827021; rs199983532; rs1327306; rs4792825; rs78361592; and rs6137844, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is haematological toxicity.

In one embodiment, the invention provides a method of screening for risk of capecitabine-induced toxicity in a subject, comprising screening the subject for the presence of at least one polymorphism selected from: rs75217739; rs139788123; rs698607; rs115143613; rs147827021; rs199983532; rs1327306; rs4792825; rs78361592; and rs6137844, and functionally equivalent variants thereof;

wherein a negative result indicates a decreased risk of developing capecitabine-induced toxicity compared to a subject which possesses said at least one polymorphism. In one embodiment, the capecitabine-induced toxicity is haematological toxicity.

In one embodiment, the invention provides a method of screening for risk of capecitabine-induced toxicity in a subject, comprising screening the subject for the presence of at least one polymorphism selected from: rs75217739; rs139788123; rs698607; rs115143613; rs147827021; rs199983532; rs1327306; rs4792825; rs78361592; and rs6137844, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is haematological toxicity.

In one embodiment, the method comprises screening the subject for 2 or more (e.g. 2, 3, 4, 5, 6, 7, 8, 9, or 10) of rs75217739; rs139788123; rs698607; rs115143613; rs147827021; rs199983532; rs1327306; rs4792825; rs78361592; and rs6137844.

Each of rs698607; rs115143613; rs147827021; rs199983532; rs1327306; rs4792825; and rs78361592 are independently associated with early onset haematological toxicity.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; and rs78361592, and functionally equivalent variants thereof; wherein the presence of said at least one polymorphism indicates an increased risk of developing capecitabine-induced toxicity compared to a subject which does not possess said at least one polymorphism. In one embodiment, the capecitabine-induced toxicity is early onset haematological toxicity.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; and rs78361592, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset haematological toxicity.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; and rs78361592, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset haematological toxicity.

Screening for rs698607; rs115143613; rs147827021; rs199983532; rs1327306; rs4792825; and rs78361592 has surprisingly been found to provide up to 91.67% sensitivity, 97.81% specificity and AUC of 97.5% as a measure of the performance of the classifier for early onset haematological toxicity.

In one embodiment, the method comprises screening the subject for 2 or more (e.g. 2, 3, 4, 5, 6 or 7) of rs698607; rs115143613; rs147827021; rs199983532; rs1327306; rs4792825; and rs78361592. In one embodiment, the method comprises screening the subject for each of: rs698607; rs115143613; rs147827021; rs199983532; rs1327306; rs4792825; and rs78361592.

Each of rs147827021 and rs6137844 are independently associated with any cycle haematological toxicity.

In one embodiment, the invention provides a method of screening for risk of capecitabine-induced toxicity in a subject, comprising screening the subject for the presence of at least one polymorphism selected from: rs147827021 and rs6137844, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle haematological toxicity.

In one embodiment, the invention provides a method of screening for risk of capecitabine-induced toxicity in a subject, comprising screening the subject for the presence of at least one polymorphism selected from: rs147827021 and rs6137844, and functionally equivalent variants thereof;

wherein a negative result indicates a decreased risk of developing capecitabine-induced toxicity compared to a subject which possesses said at least one polymorphism. In one embodiment, the capecitabine-induced toxicity is any cycle haematological toxicity.

In one embodiment, the invention provides a method of screening for risk of capecitabine-induced toxicity in a subject, comprising screening the subject for the presence of at least one polymorphism selected from: rs147827021 and rs6137844, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle haematological toxicity.

Screening for rs147827021 and rs6137844 has been found to provide up to 78.95% sensitivity, 75.66% specificity and AUC of 80.0% as a measure of the performance of the classifier for any cycle haematological toxicity.

In one embodiment, the method comprises screening the subject for both of rs147827021 and rs6137844.

Each of rs698607; rs115143613; rs147827021; rs199983532; rs1327306; rs4792825; rs78361592; and rs6137844 are independently associated with haematological toxicity.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; and rs6137844, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is haematological toxicity.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; and rs6137844, and functionally equivalent variants thereof;

wherein a negative result indicates a decreased risk of developing capecitabine-induced toxicity compared to a subject which possesses said at least one polymorphism. In one embodiment, the capecitabine-induced toxicity is haematological toxicity.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; and rs6137844, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is haematological toxicity.

In one embodiment, the method comprises screening the subject for 2 or more (e.g. 2, 3, 4, 5, 6, 7 or 8) of rs698607; rs115143613; rs147827021; rs199983532; rs1327306; rs4792825; rs78361592; and rs6137844.

Each of rs8048167; rs10654492; rs35087079; rs117686094; rs117260063; rs12231444; rs1491207661; and rs11643168 are independently associated with early onset diarrhoea toxicity.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; and rs11643168, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset diarrhoea toxicity.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; and rs11643168, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset diarrhoea toxicity.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; and rs11643168, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset diarrhoea toxicity.

In one embodiment, the method comprises screening the subject for 2 or more (e.g. 2, 3, 4, 5, 6, 7, or 8) of rs8048167; rs10654492; rs35087079; rs117686094; rs117260063; rs12231444; rs1491207661; and rs11643168. In one embodiment, the method comprises screening the subject for each of: rs8048167; rs10654492; rs35087079; rs117686094; rs117260063; rs12231444; rs1491207661; and rs11643168.

Each of rs12211900 and rs71379941 are independently associated with any cycle diarrhoea toxicity.

In one embodiment, the invention provides a method of screening for risk of capecitabine-induced toxicity in a subject, comprising screening the subject for the presence of at least one polymorphism selected from: rs12211900 and rs71379941, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle diarrhoea toxicity.

In one embodiment, the invention provides a method of screening for risk of capecitabine-induced toxicity in a subject, comprising screening the subject for the presence of at least one polymorphism selected from: rs12211900 and rs71379941, and functionally equivalent variants thereof;

wherein a negative result indicates a decreased risk of developing capecitabine-induced toxicity compared to a subject which possesses said at least one polymorphism. In one embodiment, the capecitabine-induced toxicity is any cycle diarrhoea toxicity.

In one embodiment, the invention provides a method of screening for risk of capecitabine-induced toxicity in a subject, comprising screening the subject for the presence of at least one polymorphism selected from: rs12211900 and rs71379941, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle diarrhoea toxicity.

In one embodiment, the method comprises screening the subject for both of rs12211900 and rs71379941.

Each of rs8048167; rs10654492; rs35087079; rs117686094; rs117260063; rs12231444; rs1491207661; rs11643168; rs12211900; and rs71379941 are independently associated with diarrhoea toxicity.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs12211900; and rs71379941, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is diarrhoea toxicity.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs12211900; and rs71379941, and functionally equivalent variants thereof;

wherein a negative result indicates a decreased risk of developing capecitabine-induced toxicity compared to a subject which possesses said at least one polymorphism. In one embodiment, the capecitabine-induced toxicity is diarrhoea toxicity.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs12211900; and rs71379941, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is diarrhoea toxicity.

Screening for rs8048167; rs10654492; rs35087079; rs117686094; rs117260063; rs12231444; rs1491207661; rs11643168; rs12211900; and rs71379941 has been found to provide up to 79.69% sensitivity, 87.38% specificity and AUC of 88.8% as a measure of the performance of the classifier for early onset grade 3+ diarrhoea toxicity and 59.6% sensitivity, 87.82% specificity and AUC of 78.5% as a measure of the performance of the classifier for any cycle grade 3+_diarrhoea toxicity.

In one embodiment, the method comprises screening the subject for 2 or more (e.g. 2, 3, 4, 5, 6, 7, 8, 9, or 10) of rs8048167; rs10654492; rs35087079; rs117686094; rs117260063; rs12231444; rs1491207661; rs11643168; rs12211900; and rs71379941. In one embodiment, the method comprises screening the subject for each of rs8048167; rs10654492; rs35087079; rs117686094; rs117260063; rs12231444; rs1491207661; rs11643168; rs12211900; and rs71379941.

Each of rs193272564 and rs140744481 are independently associated with early onset vomiting toxicity.

In one embodiment, the invention provides a method of screening for risk of capecitabine-induced toxicity in a subject, comprising screening the subject for the presence of at least one polymorphism selected from: rs193272564 and rs140744481, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset vomiting toxicity.

In one embodiment, the invention provides a method of screening for risk of capecitabine-induced toxicity in a subject, comprising screening the subject for the presence of at least one polymorphism selected from: rs193272564 and rs140744481, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset vomiting toxicity.

In one embodiment, the invention provides a method of screening for risk of capecitabine-induced toxicity in a subject, comprising screening the subject for the presence of at least one polymorphism selected from: rs193272564 and rs140744481, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset vomiting toxicity.

In one embodiment, the method comprises screening the subject for both rs193272564 and rs140744481.

Each of rs200676466; rs4972833; and rs148123087 are independently associated with any cycle vomiting toxicity.

In one embodiment, the invention provides a method of screening for risk of capecitabine-induced toxicity in a subject, comprising screening the subject for the presence of at least one polymorphism selected from: rs200676466; rs4972833; and rs148123087, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle vomiting toxicity.

In one embodiment, the invention provides a method of screening for risk of capecitabine-induced toxicity in a subject, comprising screening the subject for the presence of at least one polymorphism selected from: rs200676466; rs4972833; and rs148123087, and functionally equivalent variants thereof;

wherein a negative result indicates a decreased risk of developing capecitabine-induced toxicity compared to a subject which possesses said at least one polymorphism. In one embodiment, the capecitabine-induced toxicity is any cycle vomiting toxicity.

In one embodiment, the invention provides a method of screening for risk of capecitabine-induced toxicity in a subject, comprising screening the subject for the presence of at least one polymorphism selected from: rs200676466; rs4972833; and rs148123087, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle vomiting toxicity.

In one embodiment, the method comprises screening the subject for 2 or more (e.g. 2 or 3) of rs200676466; rs4972833; and rs148123087. In one embodiment, the method comprises screening the subject for each of rs200676466; rs4972833; and rs148123087.

Each of rs193272564; rs140744481; rs200676466; rs4972833; and rs148123087 are independently associated with vomiting toxicity.

In one embodiment, the invention provides a method of screening for risk of capecitabine-induced toxicity in a subject, comprising screening the subject for the presence of at least one polymorphism selected from: rs193272564; rs140744481; rs200676466; rs4972833; and rs148123087, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is vomiting toxicity.

In one embodiment, the invention provides a method of screening for risk of capecitabine-induced toxicity in a subject, comprising screening the subject for the presence of at least one polymorphism selected from: rs193272564; rs140744481; rs200676466; rs4972833; and rs148123087, and functionally equivalent variants thereof;

wherein a negative result indicates a decreased risk of developing capecitabine-induced toxicity compared to a subject which possesses said at least one polymorphism. In one embodiment, the capecitabine-induced toxicity is vomiting toxicity.

In one embodiment, the invention provides a method of screening for risk of capecitabine-induced toxicity in a subject, comprising screening the subject for the presence of at least one polymorphism selected from: rs193272564; rs140744481; rs200676466; rs4972833; and rs148123087, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is vomiting toxicity.

Screening for rs193272564, rs140744481, rs200676466, rs4972833 and rs148123087 has been found to provide up to 75% sensitivity, 99.56% specificity and AUC of 80.4% as a measure of the performance of the classifier for early onset grade 3+ vomiting toxicity and 45.45% sensitivity, 85.05% specificity and AUC of 85.5% as a measure of the performance of the classifier for any cycle grade 3+_vomiting toxicity.

In one embodiment, the method comprises screening the subject for 2 or more (e.g. 2, 3, 4, or 5) of rs193272564; rs140744481; rs200676466; rs4972833; and rs148123087. In one embodiment, the method comprises screening the subject for each of rs193272564; rs140744481; rs200676466; rs4972833; and rs148123087.

rs181841887 is independently associated with early onset hand-foot syndrome (HFS).

In one embodiment, the invention provides a method of screening for risk of capecitabine-induced toxicity in a subject, comprising screening the subject for the presence of rs181841887, and functionally equivalent variants thereof;

wherein the presence of said polymorphism indicates an increased risk of developing capecitabine-induced toxicity compared to a subject which does not possess said polymorphism. In one embodiment, the capecitabine-induced toxicity is early onset HFS.

In one embodiment, the invention provides a method of screening for risk of capecitabine-induced toxicity in a subject, comprising screening the subject for the presence of rs181841887, and functionally equivalent variants thereof;

wherein a negative result indicates a decreased risk of developing capecitabine-induced toxicity compared to a subject which possesses said polymorphism. In one embodiment, the capecitabine-induced toxicity is early onset HFS.

In one embodiment, the invention provides a method of screening for risk of capecitabine-induced toxicity in a subject, comprising screening the subject for the presence of rs181841887, and functionally equivalent variants thereof;

wherein the presence of said polymorphism indicates an increased risk of developing capecitabine-induced toxicity and a negative result indicates a decreased risk of developing capecitabine-induced toxicity. In one embodiment, the capecitabine-induced toxicity is early onset HFS.

In one embodiment, the method comprises screening the subject for rs181841887.

Each of rs79986886 and rs116448748 are independently associated with any cycle HFS.

In one embodiment, the invention provides a method of screening for risk of capecitabine-induced toxicity in a subject, comprising screening the subject for the presence of at least one polymorphism selected from: rs79986886 and rs116448748, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is any cycle HFS.

In one embodiment, the invention provides a method of screening for risk of capecitabine-induced toxicity in a subject, comprising screening the subject for the presence of at least one polymorphism selected from: rs79986886 and rs116448748, and functionally equivalent variants thereof;

wherein a negative result indicates a decreased risk of developing capecitabine-induced toxicity compared to a subject which possesses said at least one polymorphism. In one embodiment, the capecitabine-induced toxicity is any cycle HFS.

In one embodiment, the invention provides a method of screening for risk of capecitabine-induced toxicity in a subject, comprising screening the subject for the presence of at least one polymorphism selected from: rs79986886 and rs116448748, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is any cycle HFS.

In one embodiment, the method comprises screening the subject for both of rs79986886 and rs116448748.

Each of rs181841887; rs79986886; and rs116448748 are independently associated with HFS.

In one embodiment, the invention provides a method of screening for risk of capecitabine-induced toxicity in a subject, comprising screening the subject for the presence of at least one polymorphism selected from: rs181841887; rs79986886; and rs116448748, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is HFS.

In one embodiment, the invention provides a method of screening for risk of capecitabine-induced toxicity in a subject, comprising screening the subject for the presence of at least one polymorphism selected from: rs181841887; rs79986886; and rs116448748, and functionally equivalent variants thereof;

wherein a negative result indicates a decreased risk of developing capecitabine-induced toxicity compared to a subject which possesses said at least one polymorphism. In one embodiment, the capecitabine-induced toxicity is HFS.

In one embodiment, the invention provides a method of screening for risk of capecitabine-induced toxicity in a subject, comprising screening the subject for the presence of at least one polymorphism selected from: rs181841887; rs79986886; and rs116448748, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is HFS.

Screening for rs181841887; rs79986886; and rs116448748 has been found to provide up to 45.45% sensitivity, 85.05% specificity and AUC of 65.1% as a measure of the performance of the classifier for early onset grade 3+ hand foot syndrome toxicity and 41.23% sensitivity, 82.54% specificity and AUC of 62.2% as a measure of the performance of the classifier for any cycle grade 3+ hand foot syndrome toxicity.

In one embodiment, the method comprises screening the subject for 2 or more (e.g. 2 or 3) of rs181841887; rs79986886; and rs116448748. In one embodiment, the method comprises screening the subject for each of rs181841887; rs79986886; and rs116448748.

Each of rs8048167; rs10654492; rs35087079; rs117686094; rs117260063; rs12231444; rs1491207661; rs11643168; rs71313929; rs181841887; rs76211390; rs79272399; rs193272564; and rs140744481 are independently associated with early onset grade 3+toxicity of any type (“global toxicity”).

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs71313929; rs181841887; rs76211390; rs79272399; rs193272564; and rs140744481, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset grade 3+toxicity of any type.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs71313929; rs181841887; rs76211390; rs79272399; rs193272564; and rs140744481, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset grade 3+toxicity of any type.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs71313929; rs181841887; rs76211390; rs79272399; rs193272564; and rs140744481, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset grade 3+toxicity of any type.

In one embodiment, the method comprises screening the subject for 2 or more (e.g. 2, 3, 4, 5, 6, 7, 8, 9, 10, 11, 12, 13 or 14) of rs8048167; rs10654492; rs35087079; rs117686094; rs117260063; rs12231444; rs1491207661; rs11643168; rs71313929; rs181841887; rs76211390; rs79272399; rs193272564; and rs140744481. In one embodiment, the method comprises screening the subject for each of: rs8048167; rs10654492; rs35087079; rs117686094; rs117260063; rs12231444; rs1491207661; rs11643168; rs71313929; rs181841887; rs76211390; rs79272399; rs193272564; and rs140744481.

Each of rs12211900; rs71379941; rs75217739; rs139788123; rs79986886; rs116448748; rs77911328; rs145225093; rs76211390; rs117241924; rs200676466; rs4972833; and rs148123087 are independently associated with grade 3+toxicity at any cycle.

In one embodiment, the invention provides a method of screening for risk of capecitabine-induced toxicity in a subject, comprising screening the subject for the presence of at least one polymorphism selected from: rs12211900; rs71379941; rs75217739; rs139788123; rs79986886; rs116448748; rs77911328; rs145225093; rs76211390; rs117241924; rs200676466; rs4972833; and rs148123087, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is grade 3+toxicity at any cycle.

In one embodiment, the invention provides a method of screening for risk of capecitabine-induced toxicity in a subject, comprising screening the subject for the presence of at least one polymorphism selected from: rs12211900; rs71379941; rs75217739; rs139788123; rs79986886; rs116448748; rs77911328; rs145225093; rs76211390; rs117241924; rs200676466; rs4972833; and rs148123087, and functionally equivalent variants thereof;

wherein a negative result indicates a decreased risk of developing capecitabine-induced toxicity compared to a subject which possesses said at least one polymorphism. In one embodiment, the capecitabine-induced toxicity is grade 3+toxicity at any cycle.

In one embodiment, the invention provides a method of screening for risk of capecitabine-induced toxicity in a subject, comprising screening the subject for the presence of at least one polymorphism selected from: rs12211900; rs71379941; rs75217739; rs139788123; rs79986886; rs116448748; rs77911328; rs145225093; rs76211390; rs117241924; rs200676466; rs4972833; and rs148123087, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is grade 3+toxicity at any cycle.

In one embodiment, the method comprises screening the subject for 2 or more (e.g. 2, 3, 4, 5, 6, 7, 8, 9, 10, 11, 12, 13 or 14) of rs12211900; rs71379941; rs75217739; rs139788123; rs79986886; rs116448748; rs77911328; rs145225093; rs76211390; rs117241924; rs200676466; rs4972833; and rs148123087. In one embodiment, the method comprises screening the subject for each of: rs12211900; rs71379941; rs75217739; rs139788123; rs79986886; rs116448748; rs77911328; rs145225093; rs76211390; rs117241924; rs200676466; rs4972833; and rs148123087.

Each of rs8048167; rs10654492; rs35087079; rs117686094; rs117260063; rs12231444; rs1491207661; rs11643168; rs71313929; rs181841887; rs76211390; rs79272399; rs193272564; rs140744481; rs698607; rs115143613; rs147827021; rs199983532; rs1327306; rs4792825; rs78361592; rs2929329; rs35741121; rs56329496; rs117810108; rs149994224; rs11024884; and rs111378975 are independently associated with early onset grade 3+toxicity of any type (“global toxicity”).

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs71313929; rs181841887; rs76211390; rs79272399; rs193272564; rs140744481; rs698607; rs115143613; rs147827021; rs199983532; rs1327306; rs4792825; rs78361592; rs2929329; rs35741121; rs56329496; rs117810108; rs149994224; rs11024884; and rs111378975, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset grade 3+toxicity of any type.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs71313929; rs181841887; rs76211390; rs79272399; rs193272564; rs140744481; rs698607; rs115143613; rs147827021; rs199983532; rs1327306; rs4792825; rs78361592; rs2929329; rs35741121; rs56329496; rs117810108; rs149994224; rs11024884; and rs111378975, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset grade 3+toxicity of any type.

In one embodiment, the invention provides a method of screening for risk of capecitabine-induced toxicity in a subject, comprising screening the subject for the presence of at least one polymorphism selected from: rs8048167; rs10654492; rs35087079; rs117686094; rs117260063; rs12231444; rs1491207661; rs11643168; rs71313929; rs181841887; rs76211390; rs79272399; rs193272564; rs140744481; rs698607; rs115143613; rs147827021; rs199983532; rs1327306; rs4792825; rs78361592; rs2929329; rs35741121; rs56329496; rs117810108; rs149994224; rs11024884; and rs111378975, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset grade 3+toxicity of any type.

In one embodiment, the method comprises screening the subject for 2 or more (e.g. 2, 3, 4, 5, 6, 7, 8, 9, 10, 11, 12, 13, 14, 15, 16, 17, 18, 19, 20, 21, 22, 23, 24, 25, 26, 27, or 28) of rs8048167; rs10654492; rs35087079; rs117686094; rs117260063; rs12231444; rs1491207661; rs11643168; rs71313929; rs181841887; rs76211390; rs79272399; rs193272564; rs140744481; rs698607; rs115143613; rs147827021; rs199983532; rs1327306; rs4792825; rs78361592; rs2929329; rs35741121; rs56329496; rs117810108; rs149994224; rs11024884; and rs111378975. In one embodiment, the method comprises screening the subject for each of: rs8048167; rs10654492; rs35087079; rs117686094; rs117260063; rs12231444; rs1491207661; rs11643168; rs71313929; rs181841887; rs76211390; rs79272399; rs193272564; rs140744481; rs698607; rs115143613; rs147827021; rs199983532; rs1327306; rs4792825; rs78361592; rs2929329; rs35741121; rs56329496; rs117810108; rs149994224; rs11024884; and rs111378975.

Each of rs12211900; rs71379941; rs75217739; rs139788123; rs79986886; rs116448748; rs77911328; rs145225093; rs76211390; rs117241924; rs200676466; rs4972833; rs148123087; rs147827021; rs6137844; rs2420201; rs11375020; rs75504333; rs11447080; rs74380254; rs66842063; rs149994224; rs111378975; rs180742844; rs527945; and rs73039836 are independently associated with grade 3+toxicity at any cycle.

In one embodiment, the invention provides a method of screening for risk of capecitabine-induced toxicity in a subject, comprising screening the subject for the presence of at least one polymorphism selected from: rs12211900; rs71379941; rs75217739; rs139788123; rs79986886; rs116448748; rs77911328; rs145225093; rs76211390; rs117241924; rs200676466; rs4972833; rs148123087; rs147827021; rs6137844; rs2420201; rs11375020; rs75504333; rs11447080; rs74380254; rs66842063; rs149994224; rs111378975; rs180742844; rs527945; and rs73039836, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is grade 3+toxicity at any cycle.

In one embodiment, the invention provides a method of screening for risk of capecitabine-induced toxicity in a subject, comprising screening the subject for the presence of at least one polymorphism selected from: rs12211900; rs71379941; rs75217739; rs139788123; rs79986886; rs116448748; rs77911328; rs145225093; rs76211390; rs117241924; rs200676466; rs4972833; rs148123087; rs147827021; rs6137844; rs2420201; rs11375020; rs75504333; rs11447080; rs74380254; rs66842063; rs149994224; rs111378975; rs180742844; rs527945; and rs73039836, and functionally equivalent variants thereof;

wherein a negative result indicates a decreased risk of developing capecitabine-induced toxicity compared to a subject which possesses said at least one polymorphism. In one embodiment, the capecitabine-induced toxicity is grade 3+toxicity at any cycle.

In one embodiment, the invention provides a method of screening for risk of capecitabine-induced toxicity in a subject, comprising screening the subject for the presence of at least one polymorphism selected from: rs12211900; rs71379941; rs75217739; rs139788123; rs79986886; rs116448748; rs77911328; rs145225093; rs76211390; rs117241924; rs200676466; rs4972833; rs148123087; rs147827021; rs6137844; rs2420201; rs11375020; rs75504333; rs11447080; rs74380254; rs66842063; rs149994224; rs111378975; rs180742844; rs527945; and rs73039836, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is grade 3+toxicity at any cycle.

In one embodiment, the method comprises screening the subject for 2 or more (e.g. 2, 3, 4, 5, 6, 7, 8, 9, 10, 11, 12, 13, 14, 15, 16, 17, 18, 19, 20, 21, 22, 23, 24, 25, or 26) of rs12211900; rs71379941; rs75217739; rs139788123; rs79986886; rs116448748; rs77911328; rs145225093; rs76211390; rs117241924; rs200676466; rs4972833; rs148123087; rs147827021; rs6137844; rs2420201; rs11375020; rs75504333; rs11447080; rs74380254; rs66842063; rs149994224; rs111378975; rs180742844; rs527945; and rs73039836. In one embodiment, the method comprises screening the subject for each of: rs12211900; rs71379941; rs75217739; rs139788123; rs79986886; rs116448748; rs77911328; rs145225093; rs76211390; rs117241924; rs200676466; rs4972833; rs148123087; rs147827021; rs6137844; rs2420201; rs11375020; rs75504333; rs11447080; rs74380254; rs66842063; rs149994224; rs111378975; rs180742844; rs527945; and rs73039836.

Each of rs698607; rs115143613; rs147827021; rs199983532; rs1327306; rs4792825; rs78361592; rs2929329; rs35741121; rs56329496; rs117810108; rs149994224; rs11024884; and rs111378975 are independently associated with early onset grade 3+toxicity of any type (“global toxicity”).

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; rs2929329; rs35741121; rs56329496; rs117810108; rs149994224; rs11024884; and rs111378975, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is early onset grade 3+toxicity of any type.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; rs2929329; rs35741121; rs56329496; rs117810108; rs149994224; rs11024884; and rs111378975, and functionally equivalent variants thereof;

wherein a negative result indicates a decreased risk of developing capecitabine-induced toxicity compared to a subject which possesses said at least one polymorphism. In one embodiment, the capecitabine-induced toxicity is early onset grade 3+toxicity of any type.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; rs2929329; rs35741121; rs56329496; rs117810108; rs149994224; rs11024884; and rs111378975, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is early onset grade 3+toxicity of any type.

Screening for rs698607; rs115143613; rs147827021; rs199983532; rs1327306; rs4792825; rs78361592; rs2929329; rs35741121; rs56329496; rs117810108; rs149994224; rs11024884; and rs111378975 has been found to provide up to 47.89% sensitivity, 71.41% specificity AUC of 61.6% as a measure of the performance of the classifier for early onset grade 3+toxicity of any type.

In one embodiment, the method comprises screening the subject for 2 or more (e.g. 2, 3, 4, 5, 6, 7, 8, 9, 10, 11, 12, 13 or 14) of rs698607; rs115143613; rs147827021; rs199983532; rs1327306; rs4792825; rs78361592; rs2929329; rs35741121; rs56329496; rs117810108; rs149994224; rs11024884; and rs111378975. In one embodiment, the method comprises screening the subject for each of: rs698607; rs115143613; rs147827021; rs199983532; rs1327306; rs4792825; rs78361592; rs2929329; rs35741121; rs56329496; rs117810108; rs149994224; rs11024884; and rs111378975.

Each of rs12211900; rs147827021; rs6137844; rs2420201; rs11375020; rs75504333; rs11447080; rs74380254; rs66842063; rs149994224; rs111378975; rs180742844; rs527945; and rs73039836 are independently associated with grade 3+toxicity at any cycle.

In one embodiment, the invention provides a method of screening for risk of capecitabine-induced toxicity in a subject, comprising screening the subject for the presence of at least one polymorphism selected from: rs12211900; rs147827021; rs6137844; rs2420201; rs11375020; rs75504333; rs11447080; rs74380254; rs66842063; rs149994224; rs111378975; rs180742844; rs527945; and rs73039836, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is grade 3+toxicity at any cycle.

In one embodiment, the invention provides a method of screening for risk of capecitabine-induced toxicity in a subject, comprising screening the subject for the presence of at least one polymorphism selected from: rs12211900; rs147827021; rs6137844; rs2420201; rs11375020; rs75504333; rs11447080; rs74380254; rs66842063; rs149994224; rs111378975; rs180742844; rs527945; and rs73039836, and functionally equivalent variants thereof;

wherein a negative result indicates a decreased risk of developing capecitabine-induced toxicity compared to a subject which possesses said at least one polymorphism. In one embodiment, the capecitabine-induced toxicity is grade 3+toxicity at any cycle.

In one embodiment, the invention provides a method of screening for risk of capecitabine-induced toxicity in a subject, comprising screening the subject for the presence of at least one polymorphism selected from: rs12211900; rs147827021; rs6137844; rs2420201; rs11375020; rs75504333; rs11447080; rs74380254; rs66842063; rs149994224; rs111378975; rs180742844; rs527945; and rs73039836, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is grade 3+toxicity at any cycle.

Screening for rs12211900; rs147827021; rs6137844; rs2420201; rs11375020; rs75504333; rs11447080; rs74380254; rs66842063; rs149994224; rs111378975; rs180742844; rs527945; and rs73039836; has been found to provide up to 49.02% sensitivity, 65.86% specificity and AUC of 59.4% as a measure of the performance of the classifier for grade 3+toxicity at any cycle.

In one embodiment, the method comprises screening the subject for 2 or more (e.g. 2, 3, 4, 5, 6, 7, 8, 9, 10, 11, 12, 13 or 14) of rs12211900; rs147827021; rs6137844; rs2420201; rs11375020; rs75504333; rs11447080; rs74380254; rs66842063; rs149994224; rs111378975; rs180742844; rs527945; and rs73039836. In one embodiment, the method comprises screening the subject for each of: rs12211900; rs147827021; rs6137844; rs2420201; rs11375020; rs75504333; rs11447080; rs74380254; rs66842063; rs149994224; rs111378975; rs180742844; rs527945; and rs73039836.

Each of rs698607; rs115143613; rs147827021; rs199983532; rs1327306; rs4792825; rs78361592; rs2929329; rs35741121; rs56329496; rs117810108; rs149994224; rs11024884; rs111378975; rs12211900; rs6137844; rs2420201; rs11375020; rs75504333; rs11447080; rs74380254; rs66842063; rs180742844; rs527945; and rs73039836, are independently associated with any grade 3 toxicity at either early or any cycle.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; rs2929329; rs35741121; rs56329496; rs117810108; rs149994224; rs11024884; rs111378975; rs12211900; rs6137844; rs2420201; rs11375020; rs75504333; rs11447080; rs74380254; rs66842063; rs180742844; rs527945; and rs73039836, and functionally equivalent variants thereof;

wherein the presence of said at least one polymorphism indicates an increased risk of developing capecitabine-induced toxicity compared to a subject which does not possess said at least one polymorphism. In one embodiment, the capecitabine-induced toxicity is grade 3 toxicity at either early or any cycle.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; rs2929329; rs35741121; rs56329496; rs117810108; rs149994224; rs11024884; rs111378975; rs12211900; rs6137844; rs2420201; rs11375020; rs75504333; rs11447080; rs74380254; rs66842063; rs180742844; rs527945; and rs73039836, and functionally equivalent variants thereof;

wherein a negative result indicates a decreased risk of developing capecitabine-induced toxicity compared to a subject which possesses said at least one polymorphism. In one embodiment, the capecitabine-induced toxicity is grade 3 toxicity at either early or any cycle.

In one embodiment, the invention provides a method of screening for risk of capecitabine-induced toxicity in a subject, comprising screening the subject for the presence of at least one polymorphism selected from: rs698607; rs115143613; rs147827021; rs199983532; rs1327306; rs4792825; rs78361592; rs2929329; rs35741121; rs56329496; rs117810108; rs149994224; rs11024884; rs111378975; rs12211900; rs6137844; rs2420201; rs11375020; rs75504333; rs11447080; rs74380254; rs66842063; rs180742844; rs527945; and rs73039836, and functionally equivalent variants thereof;

wherein the presence of one or more of said polymorphisms indicates an increased risk of developing capecitabine-induced toxicity and a negative result indicates a decreased risk of developing capecitabine-induced toxicity. In one embodiment, the capecitabine-induced toxicity is grade 3 toxicity at either early or any cycle.

In one embodiment, the method comprises screening the subject for 2 or more (e.g. 2, 3, 4, 5, 6, 7, 8, 9, 10, 11, 12, 13, 14, 15, 16, 17, 18, 19, 20, 21, 22, 23, 24 or 25) of rs698607; rs115143613; rs147827021; rs199983532; rs1327306; rs4792825; rs78361592; rs2929329; rs35741121; rs56329496; rs117810108; rs149994224; rs11024884; rs111378975; rs12211900; rs6137844; rs2420201; rs11375020; rs75504333; rs11447080; rs74380254; rs66842063; rs180742844; rs527945; and rs73039836.

The ‘rs’ number refers to the dbSNP ID. dbSNP (“Single Nucleotide Polymorphism database”) is a public-domain archive for human single nucleotide variations, microsatellites, and small-scale insertions. dbSNP is developed and hosted by the National Center for Biotechnology Information in collaboration with the National Human Genome Research Institute.

Methods of the invention are highly predictive of capecitabine-induced toxicity. The method may include screening for any of the above-mentioned polymorphisms (individually or in combination). Thus screening may be for any of polymorphisms rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof. For example, screening may be for any of polymorphisms rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof. In another example, screening may be for any of polymorphisms rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the invention provides a method, comprising: isolating a sample, and detecting in the sample, by an assay at least one polymorphism selected from rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the invention provides a method, comprising: isolating a sample, and detecting in the sample, by an assay at least one polymorphism selected from rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof.

In one embodiment, the invention provides a method, comprising: isolating a sample, and detecting in the sample, by an assay at least one polymorphism selected from rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the invention provides a method, comprising detecting in a sample at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the invention provides a method, comprising detecting in a sample at least one polymorphism selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof.

In one embodiment, the invention provides a method, comprising detecting in a sample at least one polymorphism selected from: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the detecting comprises detecting one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the detecting comprises detecting one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof.

In one embodiment, the detecting comprises detecting one or more of: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the detecting comprises logistic regression.

In one embodiment, the sample is obtained from a cancer patient.

In one embodiment, the detecting comprises logistic regression, and the method further comprises diagnosing the subject as at risk of developing capecitabine-induced toxicity when the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof, is detected in the sample.

In one embodiment, the detecting comprises logistic regression, and the method further comprises diagnosing the subject as at risk of developing capecitabine-induced toxicity when the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof, is detected in the sample.

In one embodiment, the detecting comprises logistic regression, and the method further comprises diagnosing the subject as at risk of developing capecitabine-induced toxicity when the presence of one or more of: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof, is detected in the sample.

In one embodiment, the method comprises diagnosing the subject as at risk of developing capecitabine-induced toxicity when the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof, is detected in the sample; and administering a chemotherapeutic agent that does not comprise capecitabine or 5-FU.

In one embodiment, the method comprises diagnosing the subject as at risk of developing capecitabine-induced toxicity when the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof, is detected in the sample; and administering a chemotherapeutic agent that does not comprise capecitabine or 5-FU.

In one embodiment, the method comprises diagnosing the subject as at risk of developing capecitabine-induced toxicity when the presence of one or more of: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof, is detected in the sample; and administering a chemotherapeutic agent that does not comprise capecitabine or 5-FU.

In one embodiment, the method further comprises administering a chemotherapeutic agent that does not comprise capecitabine or 5-FU when the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof, is detected in the sample.

In one embodiment, the method further comprises administering a chemotherapeutic agent that does not comprise capecitabine or 5-FU when the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof, is detected in the sample.

In one embodiment, the method further comprises administering a chemotherapeutic agent that does not comprise capecitabine or 5-FU when the presence of one or more of: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof, is detected in the sample.

In one embodiment, the invention provides a method comprising obtaining a sample from a patient; and exposing the sample to a reagent capable of detecting in the sample the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

In one embodiment, the invention provides a method comprising obtaining a sample from a patient; and exposing the sample to a reagent capable of detecting in the sample the presence of one or more of: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof.

In one embodiment, the invention provides a method comprising obtaining a sample from a patient; and exposing the sample to a reagent capable of detecting in the sample the presence of one or more of: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

The invention also provides a composition for detecting the presence of one or more polymorphisms in a sample obtained from a patient, comprising: a) the sample, and (b) means for amplifying the one or more polymorphisms in the sample, wherein the one or more polymorphisms are selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836, and functionally equivalent variants thereof.

The invention also provides a composition for detecting the presence of one or more polymorphisms in a sample obtained from a patient, comprising: a) the sample, and (b) means for amplifying the one or more polymorphisms in the sample, wherein the one or more polymorphisms are selected from: rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; and rs181841887, and functionally equivalent variants thereof.

The invention also provides a composition for detecting the presence of one or more polymorphisms in a sample obtained from a patient, comprising: a) the sample, and (b) means for amplifying the one or more polymorphisms in the sample, wherein the one or more polymorphisms are selected from: rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945, and rs73039836, and functionally equivalent variants thereof.

Methods of the invention represent a significant improvement on methods based on using commercially available kits.

The subject may be a cancer patient. The patient may have a solid tumour cancer such as colorectal cancer (CRC), breast cancer, or gastrointestinal cancer. The subject may be undergoing (or have undergone) chemotherapy with 5-FU. Alternatively, the method may be carried out on a subject who is about to undergo chemotherapy with 5-FU, such as a subject who has been identified as a candidate for chemotherapy with 5-FU. Chemotherapy with 5-FU includes chemotherapy with capecitabine.

Chemotherapy with 5-FU may be a 5-FU monotherapy, such as capecitabine monotherapy. The term “chemotherapy with 5-FU” however includes any therapy based on 5-FU (typically comprising capecitabine therapy) either alone or in combination with one or more other agents, e.g. FOLFOX, XELOX, or FOLFIRI.

Screening for the presence of polymorphisms of the invention may be carried out on a sample from the subject. This sample may be a fluid sample, such as a saliva, blood, serum or plasma sample. This sample may be a solid sample, such as a sample from a biopsy.

Screening for polymorphisms with equivalent functional effects to rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and/or rs73039836 is also included within the scope of this invention. Such variants may be screened for using available gene sequencing or direct functional assays as are known in the technical field.

The method of the invention permits close monitoring of a subject in which one or more of the above-mentioned polymorphisms are present and who is therefore at increased risk of toxicity. The method of the invention may accordingly include a step of monitoring the subject for symptoms of capecitabine-induced toxicity in the event of a positive result, i.e. where one or more of the above-mentioned polymorphisms is detected.

The capecitabine-induced toxicity risk may be high-grade toxicity, i.e. grade 3+ in accordance with the NCI Common Toxicity Criteria for Adverse Events (CTCAE) version 3.0. The high-grade toxicity may be global toxicity. The high-grade toxicity may include diarrhoea, haematological toxicity, nausea and vomiting, mucositis/stomatitis, myelosuppression, neutropaenia, thrombocytopaenia, and hand-foot syndrome (HFS).

Polymorphisms of the invention may be identified using a score test, which tests for independent effects of variants within a region. For example, polymorphisms of the invention may be identified using a genetic risk score test. This test may comprise performing logistic regression with toxicity for the number of high-risk alleles carried by an individual and correspondingly assigning the subject a genetic score (e.g. from 0 to 4). The following is an example, genetic risk score=ΣβiNi, where βi is the beta coefficient of the ith SNP significantly associated with global toxicity in a logistic regression model, and Ni is the number of harmful alleles carried by that individual at that locus.

Polymorphisms may also be identified using a group test.

The invention also provides one or more reagents capable of detecting the presence of the above-mentioned polymorphisms e.g. in a sample obtained from a patient, for use in the method of the invention.

The above-mentioned reagents may be present in a kit. Accordingly, the invention provides a kit comprising one or more of the above-mentioned reagents capable of detecting the presence of the above-mentioned polymorphisms.

Screening for the presence of at least one polymorphism may comprise sequencing methods known in the art such as PCR.

### FIGURES

FIG. 1: Receiver operator curve (ROC) analysis of subsets of the novel SNPs using QUASAR 2 data.
(a): 7 SNPs associated with early onset grade 3+mucositis toxicity were used in the ROC analysis of this phenotype (these 7 SNPs were rs2929329; rs35741121; rs56329496; rs117810108; rs149994224; rs11024884 and rs111378975). (b): 10 SNPs associated with any (early or all) cycle mucositis grade 3+toxicity were used in the ROC analysis of this phenotype (these 10 SNPs were rs2420201; rs11375020; rs75504333; rs11447080; rs74380254; rs66842063; rs149994224; rs111378975; rs180742844 and rs527945). (c): 7 SNPs associated with early onset haematological toxicity were used in the ROC analysis of this phenotype (these 7 SNPs were rs698607; rs115143613; rs147827021; rs199983532; rs1327306; rs4792825 and rs78361592). (d): 2 SNPs associated with any (early or all) cycle haematological toxicity were used in the ROC analysis of this phenotype (these 2 SNPs were rs147827021 and rs6137844). (e): 14 SNPs associated with early onset grade 3+toxicity of any type were used in the ROC analysis of this phenotype (these 14 SNPs were rs698607; rs115143613; rs147827021; rs199983532; rs1327306; rs4792825; rs78361592; rs2929329; rs35741121; rs56329496; rs117810108; rs149994224; rs11024884 and rs111378975. (f): 14 SNPs (partially overlapping with the early onset set (FIG. 1(e)) associated with grade 3+toxicity at any cycle were used in the ROC analysis of this phenotype (these 14 SNPs were rs12211900; rs147827021; rs6137844; rs2420201; rs11375020; rs75504333; rs11447080; rs74380254; rs66842063; rs149994224; rs111378975; rs180742844; rs527945 and rs73039836).

FIG. 2: Receiver operator curve (ROC) analysis of subsets of the novel SNPs identified using the expected genotype dosages method for analysing QUASAR 2 data 5 SNPs (rs76211390; rs79272399; rs77911328; rs145225093; and rs117241924) associated with early onset or any cycle grade 3+mucositis toxicity were used in the ROC analysis of early onset grade 3+mucositis toxicity (a) and all cycle grade 3+mucositis (b). 2 SNPs (rs75217739 and rs139788123) associated with all cycle grade 3+ haematological toxicity were used in the ROC analysis of early onset grade 3+ haematological toxicity (c) and all cycle grade 3+ haematological toxicity (d). 10 SNPs (rs8048167, rs10654492, rs35087079, rs117686094, rs117260063, rs12231444, rs1491207661, rs11643168, rs12211900 and rs71379941) associated with early onset or any cycle grade 3+ diarrhoea toxicity were used in the ROC analysis of early onset grade 3+ diarrhoea toxicity (e) and all cycle grade 3+ diarrhoea (f) toxicity. 5 SNPs (rs193272564, rs140744481, rs200676466, rs4972833, rs148123087) associated with early onset or all cycle grade 3+ vomiting toxicity were used in the ROC analysis of early onset grade 3+ vomiting (g) and all cycle grade 3+ vomiting toxicity (h). 3 SNPs (rs181841887, rs79986886, rs116448748) associated with early onset or all cycle grade 3+ hand-foot syndrome were used in the ROC analysis of early onset grade 3+ hand foot syndrome (i) and all cycle grade 3+ hand foot syndrome (j). 26 SNPs associated with early onset grade 3+toxicity of any type or all cycle grade 3+toxicity of any type were used in the ROC analysis of early onset grade 3+toxicity of any type (k) and all cycle grade 3+toxicity of any type (l). These 26 SNPs were rs8048167; rs10654492; rs35087079; rs117686094; rs117260063; rs12231444; rs1491207661; rs11643168; rs193272564; rs140744481; rs76211390; rs79272399; rs71313929; rs12211900; rs71379941; rs75217739; rs139788123; rs79986886; rs116448748; rs77911328; rs145225093; rs117241924; rs200676466; rs4972833; rs148123087; rs181841887.

### EXAMPLES

The invention will be further clarified by the following examples, which are intended to be purely exemplary of the invention and are in no way limiting.

### Example 1

### Methods

i. Patient and Study Characteristics

The QUASAR2 study (http://www.octo-oxford.org.uk/alltrials/infollowup/q2.html; http://www.controlled-trials.com/ISRCTN45133151/) is a phase III randomised controlled trial of capecitabine (Xeloda) (1250 mg/m2 twice daily d1-14 every 3 weeks, total of 8 cycles)+/−bevacizumab (7.5 mg/kg every three weeks) following resection of stage II/III CRC. Patients were entered into the study between July 2005 and December 2011 at 123 UK and 81 non-UK sites. Of 1119 patients with blood collected as of July 2010, 1046 were selected for study based on availability of clinical data and informed consent.

ii. Assessment of Capecitabine Toxicity

Adverse events were graded following each treatment cycle using the NCI Common Toxicity Criteria for Adverse Events (CTCAE) version 3.0. Common capecitabine-induced toxicities—diarrhoea, nausea and vomiting, mucositis/stomatitis, neutropaenia, thrombocytopaenia, and HFS—were analysed individually and also in combination as “global” toxicity. Adverse events were categorised as low (CTCAE grade 0/1/2) and high (CTCAE grade 3/4/5 at any treatment cycle). Hypertension and proteinuria were clearly related to bevacizumab (˜10-fold higher incidence) and were not included in the analysis. The incidence of capecitabine-induced toxicities did not differ materially between the two study arms and these were combined for analysis.

iii. Genome-Wide Association Studies

The Inventors performed genome-wide association studies (GWAS) to identify novel genetic markers associated with serious capecitabine toxicity occurring either in cycles 1 and 2 (denoted “early onset”) or across all cycles (denoted “all cycle”) of treatment with this drug. GWAS enable an agnostic search of the human genome for the detection of novel genetic variants (“What are genome-wide association studies (GWAS)?” [Internet]. EMBL-EBI Train Online. 2020).

Genome wide SNP genotypes and per treatment cycle toxicity data was available for 930 patients with stage II and III colorectal cancer treated with capecitabine in the QUASAR2 trial. This data has been previously described (Rosmarin D, et al. Gut. (2015) January; 64(1):111-20 Epub 2014 Mar. 19; and Palles C, et al. Cancers (Basel). (2021) Mar. 24; 13(7):1497), incorporated herein by reference. Genotype data was imputed using IMPUTE2 and the UK10K and 1000 genomes merged reference panels. Common Terminology Criteria for Adverse Events (CTCAE) v3.0 was used to grade toxicity events. Patients with ≥grade 3 toxicities in the first two treatment cycles and across all cycles were coded as cases for analysis. Binary coded variables were generated for capecitabine-induced toxicities: diarrhoea, haematological, hand-foot syndrome, mucositis and vomiting. The mucositis toxicity variable captured cases with mucositis and/or stomatitis and haematological toxicity consisted of neutropenia and/or thrombocytopenia cases. A global toxicity phenotype was also analysed with cases having any grade 3 event of one of the aforementioned toxicities. The study was powered at 80% to detect variants with MAF>10% having effect sizes≥2. Association testing was performed using SNPTEST v2.5. The two QUASAR2 trial arms (capecitabine alone and capecitabine plus bevacizumab) were analysed separately and then meta-analysed using META v1.7. The outputs were filtered to retain variants with high quality imputation (INFO score>=0.8), hardy-Weinberg equilibrium P>1×10⁻⁶, minor allele frequency (MAF)>1% in cases and controls in each arm, GWAS significance (P<5×10⁻⁸) or suggestive significance (P<5×10⁻⁷) and P heterogeneity >0.05. Gene annotation of the outputs was performed using Variant Effect Predictor (VEP) and ANNOVAR to determine the impact of each variant and distance to the nearest gene. Top independent SNPs were identified from multi-SNP-based conditional and joint association (COJO) analysis using Genome-wide Complex Trait Analysis (GCTA). V1.93.2. Receiver operator curve (ROC) analysis was performed in statistical software R.

### Results

The number of patients from QUASAR 2 with CTCAE grade 0 to 2 and grade 3 to 4 capecitabine-induced toxicities during their first two cycles of treatment is shown in Table 1.

TABLE 1
Patients with ‘early onset’ capecitabine-induced
toxicities (individual and global) in QUASAR 2. Missing
data from each toxicity variable is coded as ‘NA’.

| | Cycles 1 & 2 (n = 930) | | |
|---|---|---|---|
| Toxicity | Grade 0-2 (n, %) | Grade 3-4 (n, %) | NA (n, %) |
| Diarrhoea | 864 (92.9) | 64 (6.9) | 2 (0.2) |
| Haematological | 915 (98.4) | 12 (1.3) | 3 (0.3) |
| Hand-foot syndrome | 850 (91.4) | 77 (8.3) | 3 (0.3) |
| Mucositis | 918 (98.7) | 10 (1.1) | 2 (0.2) |
| Vomiting | 918 (98.7) | 8 (0.9) | 4 (0.4) |
| Global | 787 (84.6) | 142 (15.3) | 1 (0.1) |

A total of 25 independent SNPs were identified to be associated with either early onset (cycle 1 or cycle 2) capecitabine-induced ≥grade 3 toxicities and/or ≥grade 3 toxicity events experienced across all treatment cycles (Table 2) at genome-wide significance (P<5×10⁻⁸). These include rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836.

Each of the 25 independent SNPs have a minor allele frequency (MAF)>5%. Of these 25 SNPs, three were associated with both early onset and all cycle ≥grade 3 events (rs147827021; rs149994224 and rs111378975). Joint and conditional association testing confirmed that the 25 SNPs identified in this example are independent of one another.

TABLE 2
Top independent SNPs from GWAS of early onset and all cycles capecitabine toxicity from conditional and joint (‘COJO’) analysis.

| Early/All | | | | Toxicity | | | | | | |
|---|---|---|---|---|---|---|---|---|---|---|
| cycles | Toxicity | chr | SNP | risk allele | rsIDs | freq | OR | Lower CI | Upper CI | P value |
| All | Diarrhoea | 6 | 6: 37510161_T_C | C | rs12211900 | 0.11 | 4.25 | 2.64 | 6.84 | 2.73E−09 |
| Early | Haematological | 2 | 2: 191052117_A_G | G | rs698607 | 0.07 | 742 | 113 | 4861 | 5.49E−12 |
| Early | Haematological | 3 | 3: 187661895_A_T | T | rs115143613 | 0.06 | 562 | 66 | 4734 | 5.68E−09 |
| *Early | Haematological | 7 | 7: 121686924_TG_T | T | rs147827021 | 0.07 | 266 | 39 | 1783 | 8.77E−09 |
| Early | Haematological | 10 | 10: 133335072_ACAGT_A | A | rs199983532 | 0.06 | 223 | 33 | 1500 | 2.64E−08 |
| Early | Haematological | 13 | 13: 27670283_T_C | T | rs1327306 | 0.93 | 0.01 | 0.0017 | 0.05 | 2.49E−08 |
| Early | Haematological | 17 | 17: 43919681_A_G | G | rs4792825 | 0.10 | 74 | 16.47 | 331.85 | 1.96E−08 |
| Early | Haematological | 20 | 20: 56132320_A_G | G | rs78361592 | 0.05 | 426 | 53 | 3441 | 1.31E−08 |
| *All | Haematological | 7 | 7: 121686924_TG_T | T | rs147827021 | 0.07 | 110 | 26.75 | 455.79 | 7.84E−11 |
| All | Haematological | 20 | 20: 23130488_G_A | A | rs6137844 | 0.08 | 48 | 12.63 | 182.48 | 1.33E−08 |
| Early | Mucositis | 1 | 1: 218769034_T_C | T | rs2929329 | 0.85 | 0.02 | 0.004 | 0.07 | 2.04E−08 |
| Early | Mucositis | 6 | 6: 90109696_G_A | A | rs35741121 | 0.09 | 465 | 72 | 3004 | 1.11E−10 |
| Early | Mucositis | 6 | 6: 135530039_C_A | A | rs56329496 | 0.10 | 275 | 45 | 1670 | 1.05E−09 |
| Early | Mucositis | 7 | 7: 130434431_T_G | G | rs117810108 | 0.05 | 2202 | 205 | 23677 | 2.13E−10 |
| *Early | Mucositis | 10 | 10: 85622866_AATAT_A | A | rs149994224 | 0.12 | 181 | 32.33 | 1010.32 | 3.23E−09 |
| Early | Mucositis | 11 | 11: 18950092_A_G | G | rs11024884 | 0.09 | 349 | 43.72 | 2787.34 | 3.31E−08 |
| *Early | Mucositis | 12 | 12: 120838305_C_T | T | rs111378975 | 0.05 | 9598 | 812 | 113409 | 3.4E−13 |
| All | Mucositis | 2 | 2: 72356370_G_A | A | rs2420201 | 0.06 | 255 | 3.66 | 7.42 | 7.34E−09 |
| All | Mucositis | 4 | 4: 84708637_A_AT | A | rs11375020 | 0.92 | 0.01 | 0.002 | 0.05 | 8.61E−09 |
| All | Mucositis | 6 | 6: 169827440_G_C | C | rs75504333 | 0.08 | 82.65 | 17.64 | 387.33 | 2.12E−08 |
| All | Mucositis | 6 | 6: 90110314_T_TA | TA | rs11447080 | 0.21 | 27.37 | 10.16 | 73.70 | 5.83E−11 |
| All | Mucositis | 7 | 7: 77324832_G_C | C | rs74380254 | 0.07 | 96.24 | 19.71 | 469.92 | 1.66E−08 |
| All | Mucositis | 10 | 10: 53605694_TTGAG_T | T | rs66842063 | 0.09 | 101 | 20 | 509 | 2.27E−08 |
| *All | Mucositis | 10 | 10: 85622866_AATAT_A | A | rs149994224 | 0.12 | 58.62 | 14.47 | 237.54 | 1.18E−08 |
| *All | Mucositis | 12 | 12: 120838305_C_T | T | rs111378975 | 0.05 | 410 | 52 | 3197 | 9.31E−09 |
| All | Mucositis | 18 | 18: 14069319_A_G | G | rs180742844 | 0.07 | 401 | 59 | 2736 | 9.38E−10 |
| All | Mucositis | 21 | 21: 40904207_C_T | C | rs527945 | 0.94 | 0.0007 | 0.0001 | 0.005 | 3.31E−14 |
| All | Vomiting | 12 | 12: 4698627_C_G | G | rs73039836 | 0.07 | 1265 | 202 | 7914 | 2.28E−14 |

*SNPs associated with mucositis or haematological toxicity variable both during the first two cycles and when analysed across all cycles.
“Chr” = chromosome; “Freq” = frequency; “OR” = odds ratio; “Lower CI” = lower confidence interval; “Upper CI” = upper confidence interval. “Toxicity risk allele” = allele associated with toxicity risk (e.g. for rs12211900, 6: 37510161_C is associated with toxicity risk).

TABLE 3
Annovar and VEP gene annotations for top independent SNPs

| rsIDs | Gene annotation from Annovar | Gene annotation from VEP |
|---|---|---|
| rs12211900 | LINC02520(dist = 6105, | RP1-153P14.5 |
| | MIR4462(dist = 12980) | |
| rs698607 | C2orf88 | C2orf88 |
| rs115143613 | BCL6(dist = 198382), LINC01991(dist = 14668) | RP11-44H4.1 |
| rs147827021 | PTPRZ1 | PTPRZ1 |
| rs199983532 | TCERG1L(dist = 225088), LINC01164 | NA |
| | (dist = 269662) | |
| rs1327306 | USP12 | USP12 |
| rs4792825 | LINC02210-CRHR1(dist = 6487), MAPT- | SPPL2C |
| | AS1(dist = 1041) | |
| rs78361592 | CTCFL(dist = 31612), | PCK1 |
| | PCK1(dist = 3817) | |
| rs147827021 | PTPRZ1 | PTPRZ1 |
| rs6137844 | LINC00656(dist = 17215, | NA |
| | NXT1(dist = 200885) | |
| rs2929329 | MIR548F3 | NA |
| rs35741121 | RRAGD | RRAGD |
| rs56329496 | MYB | MYB |
| rs117810108 | KLF14 (dist = 15571), MIR29A (dist = 127075) | NA |
| rs149994224 | LOC105378397(dist = 186021), | NA |
| | GHITM(dist = 276319) | |
| rs11024884 | PTPN5(dist = 135824), | NA |
| | MRGPRX1(dist = 5268) | |
| rs111378975 | MSI1(dist = 31322), COX6A1(dist = 37588) | RP1-166H1.2 |
| rs2420201 | CYP26B1(NM_001277742:c. *2986T > C, NM_01 | CYP26B1 |
| | 9885:c. *2986T > C) | |
| rs11375020 | GPAT3(dist = 181610), | NA |
| | LOC101928978 | |
| | (dist = 180598) | |
| rs75504333 | THBS2(dist = 173231), | RP1-137D17.2 |
| | WDR27(dist = 29863) | |
| rs11447080 | RRAGD | RRAGD |
| rs74380254 | APTR | RSBN1L |
| rs66842063 | PRKG1 | PRKG1 |
| rs149994224 | LOC105378397 | NA |
| | (dist = 186021), | |
| | GHITM(dist = 276319) | |
| rs111378975 | MSI1(dist = 31322), | RP1-166H1.2 |
| | COX6A1(dist = 37588) | |
| rs180742844 | MC2R(dist = 153613), | ZNF519 |
| | ZNF519(dist = 30623) | |
| rs527945 | WRB- SH3BGR (dist = 16774), B3GALT5-AS1 | AF121897.4 |
| | (dist = 64868) | |
| rs73039836 | DYRK4(dist = 611) | DYRK4 |

To provide some measure of the sensitivity of a test including these SNPs for the prediction of early and all cycle capecitabine induced ≥grade 3 toxicity receiver operator curve analysis was performed using QUASAR 2 samples.

FIG. 1 shows the area under the curve, sensitivity and specificity of tests including SNPs specifically associated with haematological toxicity or mucositis and/or stomatitis. The performance of the full set of SNPs at predicting global toxicity was also analysed this way.

### Mucositis Toxicity

FIG. 1(a) provides ROC analysis for 7 SNPs associated with early onset grade 3+mucositis toxicity (rs2929329; rs35741121; rs56329496; rs117810108; rs149994224; rs11024884 and rs111378975). Results when these 7 SNPs were included were AUC 98.7% (95% CI: 96.7%-100.0%), specificity of 90.20 and sensitivity of 100%, at a threshold of 0.55 for early onset mucositis.

FIG. 1 (b) provides ROC analysis for 10 SNPs associated with any cycle grade 3+mucositis+toxicity (rs2420201; rs11375020; rs75504333; rs11447080; rs74380254; rs66842063; rs149994224; rs111378975; rs180742844 and rs527945). Results when these 10 SNPs were included were AUC 99.7% (95% CI: 99.7%-100.0%), specificity of 98.36 and sensitivity of 100%, at a threshold of −0.23 for any cycle mucositis.

When all 15 SNPs associated with mucositis toxicity were included, results were AUC 99.8% (95% CI: 99.4%-100%), specificity of 98.26% and sensitivity of 100% at threshold of −0.26 for early onset mucositis, and AUC 99.5% (95% CI: 98.8%-100%), specificity of 95.41%, sensitivity of 100% at threshold of −0.44 of any cycle mucositis.

### Haematological Toxicity

FIG. 1(c) provides ROC analysis for 7 SNPs associated with early onset haematological toxicity (rs698607; rs115143613; rs147827021; rs199983532; rs1327306; rs4792825 and rs78361592). Results when these 7 SNPs were included were AUC 97.5% (95% CI: 93.9%-100.0%), specificity of 97.81 and sensitivity of 91.67%, at a threshold of 0.47 for early onset haematological toxicity.

FIG. 1(d) provides ROC analysis for 2 SNPs associated with any cycle haematological toxicity (rs147827021 and rs6137844). Results when these 2 SNPs were included were AUC 80.0% (95% CI: 68.9%-91.2%), specificity of 75.66% and sensitivity of 78.95%, at a threshold of 0.90 for any cycle haematological toxicity.

When all 8 SNPs associated with haematological toxicity were included, results were AUC 98.7% (95% CI: 97-100%), specificity of 90.27%, sensitivity of 100% at threshold of 0.11 for early onset haematological toxicity, and AUC 90.9% (95% CI: 81.7%-100%), specificity of 84.91% sensitivity of 89.47% at threshold of 0.0009 for any cycle haematological toxicity.

### Global Toxicity

FIG. 1(e) provides ROC analysis for 14 SNPs associated with early onset grade 3+toxicity of any type (rs698607; rs115143613; rs147827021; rs199983532; rs1327306; rs4792825; rs78361592; rs2929329; rs35741121; rs56329496; rs117810108; rs149994224; rs11024884 and rs111378975). Results when these 14 SNPs were included were AUC 61.6% (95% CI: 56.2%-66.9%), specificity of 71.41% and sensitivity of 47.89%, at a threshold of −0.01 for early onset grade 3+toxicity.

FIG. 1(f) provides ROC analysis for 14 SNPs (partially overlapping with the early onset set (FIG. 1(e)) associated with grade 3+toxicity at any cycle (rs12211900; rs147827021; rs6137844; rs2420201; rs11375020; rs75504333; rs11447080; rs74380254; rs66842063; rs149994224; rs111378975; rs180742844; rs527945 and rs73039836). Results when these 14 SNPs were included were AUC 59.4% (95% CI: 55.6%-63.3%), specificity of 65.86% and sensitivity of 49.02%, at a threshold of −0.003 for any cycle grade 3+toxicity.

When all SNPs associated with any grade 3 toxicity at either early or any cycle were used results were AUC 63% (95% CI: 57.8%-68.2%), specificity of 60.23%, sensitivity of 60.56% at threshold of −0.06 for grade 3+ early onset toxicity, and AUC 61.3% (95% CI: 57.5%-65.2%), specificity of 75.20%, sensitivity of 41.23% at threshold of −0.02 for any cycle global grade 3+toxicity.

The analysis suggests that the polymorphisms may also be useful in predicting toxicity in other 5-FU monotherapy regimens, but these regimens are used uncommonly.

### Example 2

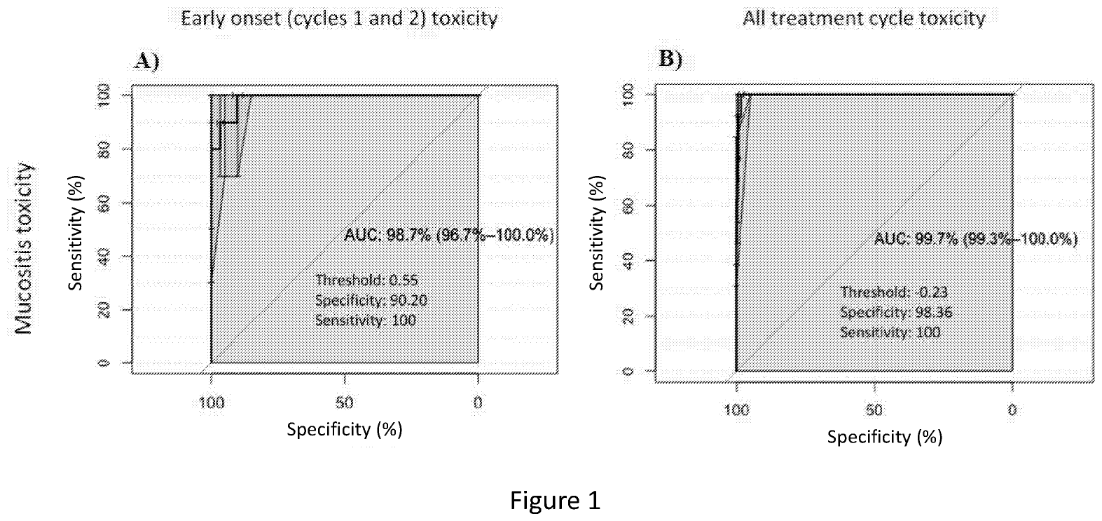

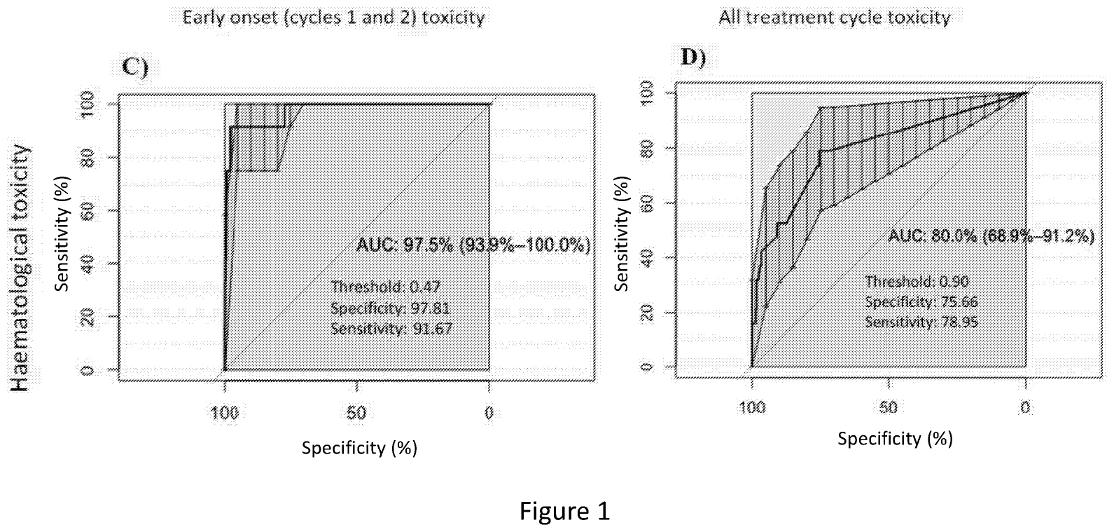

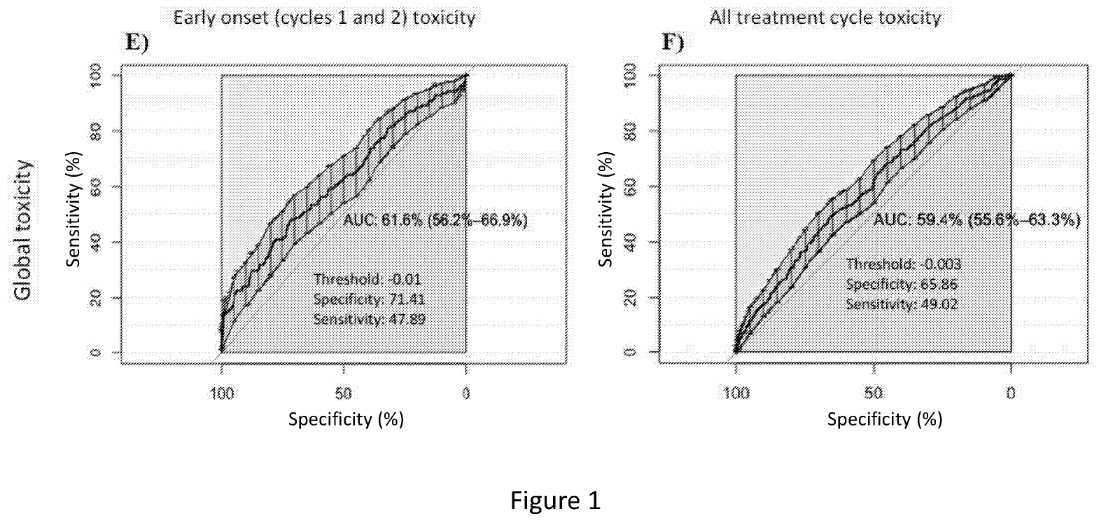

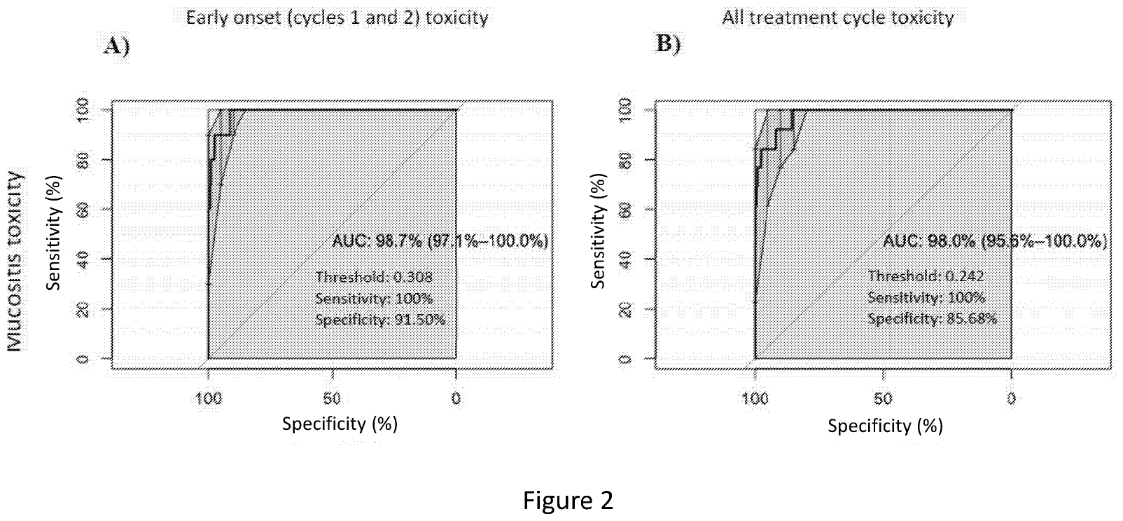

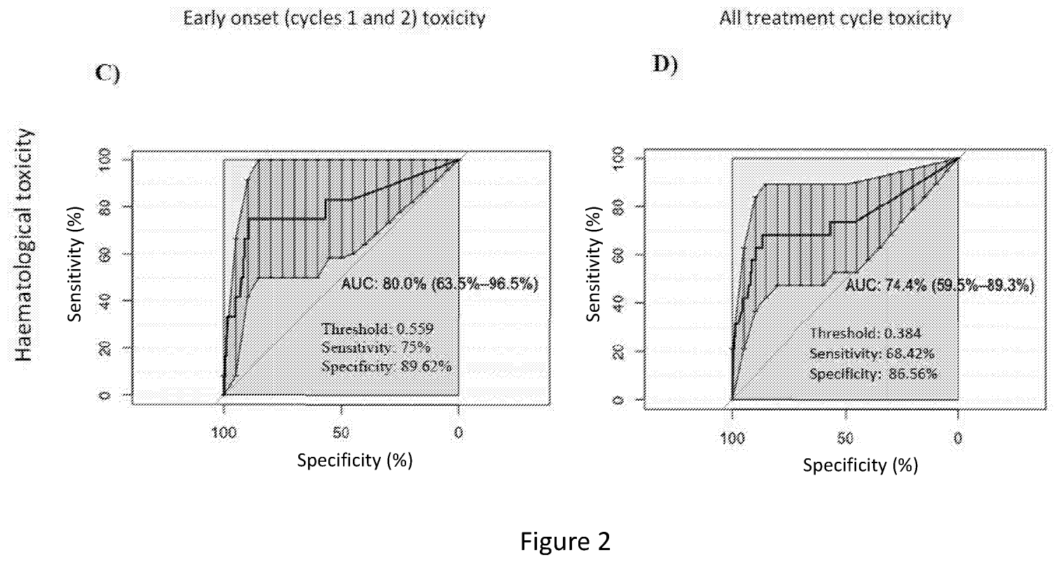

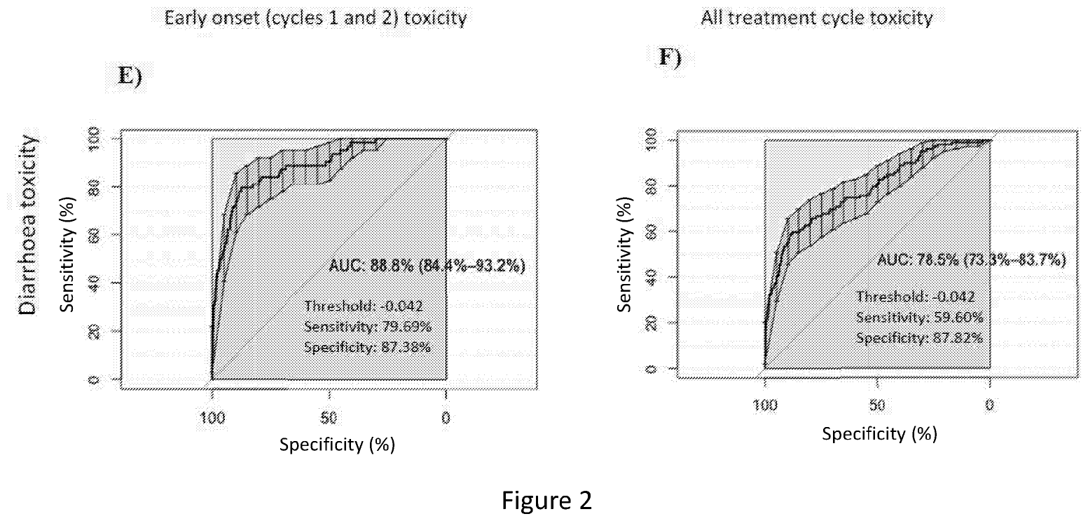

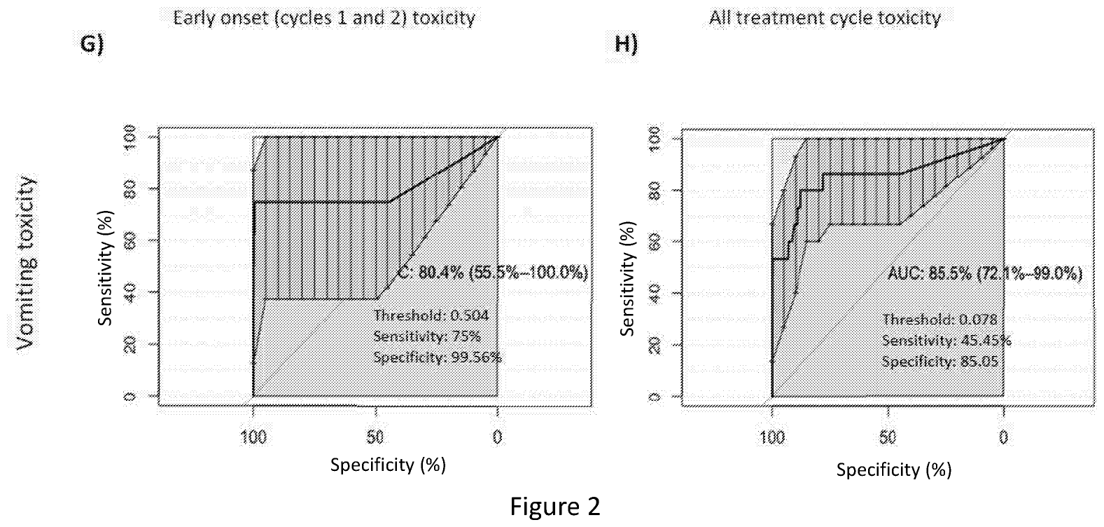

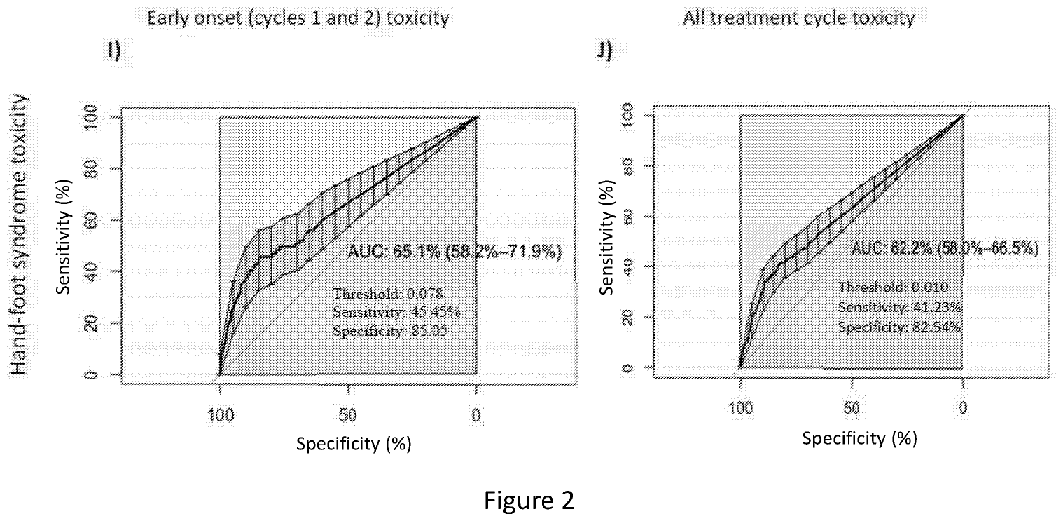

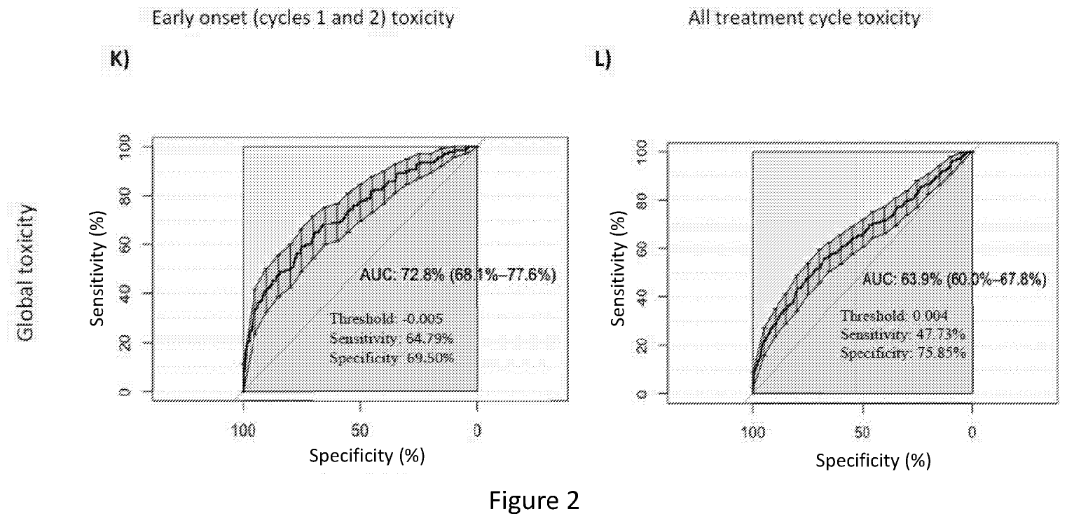

The analysis described in Example 1 was repeated using a robust expected-genotype dosage method to handle genotypes. Using this method, an additional 25 independent SNPs were identified as being associated with either early onset (cycle 1 or cycle 2) capecitabine-induced ≥grade 3 toxicities and/or ≥grade 3 toxicity events experienced across all treatment cycles (Table 4) at or close to genome-wide significance (P<5×10⁻⁷). The sensitivity and specificity of tests including various combinations of these SNPs were calculated (FIG. 2).

FIG. 2 shows the area under the curve, sensitivity and specificity of tests including SNPs identified using the expected genotype dosage method of handling genotype data specifically associated with haematological toxicity, mucositis and/or stomatitis, diarrhoea, vomiting and hand-foot syndrome. The performance of the full set of SNPs at predicting global toxicity was also analysed this way.

### Mucositis Toxicity

FIG. 2(a) provides ROC analysis for 5 SNPs (rs76211390, rs79272399, rs77911328, rs145225093 and rs117241924) and early onset grade 3+mucositis toxicity and FIG. 2(b) provides ROC analysis for the same 5 SNPs and any cycle grade 3+mucositis toxicity. Results when these 5 SNPs were included were AUC 98.7% (95% CI: 97.1%-100.0%), specificity of 91.50 and sensitivity of 100%, at a threshold of 0.308 for early onset grade 3+mucositis and AUC 98.0% (95% CI 95.6-100%), 85.65% specificity, 100% sensitivity at a threshold of 0.242 for ≥grade 3 mucositis experienced across all treatment cycles.

### Haematological Toxicity

FIG. 2(c) provides ROC analysis for 2 SNPs (rs75217739 and rs139788123) and early onset grade 3+ haematological toxicity and FIG. 2(d) provides ROC analysis for the same 2 SNPs (rs75217739 and rs139788123) and any cycle grade 3+ haematological toxicity. Results when these 2 SNPs were included were AUC 80.0% (95% CI: 63.5-96.5%), specificity of 98.62 and sensitivity of 75%, at a threshold of 0.559 for early onset haematological toxicity and AUC 74.4% (95% CI: 59.5%-89.3%), specificity of 86.56% and sensitivity of 68.42%, at a threshold of 0.384 for any cycle haematological toxicity.

### Diarrhoea Toxicity

FIG. 2(e) provides ROC analysis for 10 SNPs (rs8048167, rs10654492, rs35087079, rs117686094, rs117260063, rs12231444, rs1491207661, rs11643168, rs12211900 and rs71379941) and early onset grade 3+ diarrhoea and FIG. 2(f) provides ROC analysis for 10 SNPs (rs8048167, rs10654492, rs35087079, rs117686094, rs117260063, rs12231444, rs1491207661, rs11643168, rs12211900 and rs71379941) and any cycle grade 3+ diarrhoea. Results when these 10 SNPs were included were AUC 88.8% (95% CI 84.4-93.2%), specificity 87.3, sensitivity 79.69 at a threshold of −0.042 for early onset grade 3+ diarrhoea toxicity and AUC 78.5% (95% CI 73.3-83.7%), specificity 87.82, sensitivity 59.60 at a threshold of −0.042 for any cycle grade 3+ diarrhoea toxicity.

### Vomiting

FIG. 2(g) provides ROC analysis for 5 SNPs (rs193272564, rs140744481, rs200676466, rs4972833 and rs148123087) and early onset grade 3+ vomiting and FIG. 2(h) provides ROC analysis for the same SNPs and any cycle grade 3+ vomiting. Results when these 5 SNPs were included were AUC 80.4% (95% CI 55.5-100%), specificity 99.56, sensitivity 75% at a threshold of 0.504 for early onset grade 3+ vomiting and AUC 85.5% (72.1-99.0%), specificity 85.05%, sensitivity 45.45% at a threshold of 0.078 for any cycle grade 3+ vomiting.

### Hand Foot Syndrome

FIG. 2(i) provides ROC analysis for 3 SNPs (rs181841887, rs79986886 and rs116448748) and early onset grade 3+ hand foot syndrome and FIG. 2(j) provides ROC analysis for the same three SNPs and any cycle grade 3+ hand foot syndrome. Results when these 5 SNPs were included were AUC 65.1% (95% CI 58.2-71.9%), specificity 85.05%, sensitivity 45.45% at a threshold of 0.078 for early onset grade 3+ hand foot syndrome and AUC 62.2% (95% CI 58.0%-66.5%), specificity 82.54%, sensitivity 41.23% at a threshold of 0.010 for any cycle grade 3+ hand foot syndrome.

### Global Toxicity

FIG. 2(k) provides ROC analysis for all 26 SNPs associated with early onset grade 3+toxicity of any type or any cycle grade 3+toxicity of any type (rs8048167, rs10654492, rs35087079, rs117686094, rs117260063, rs12231444, rs1491207661, rs11643168, rs193272564, rs140744481, rs76211390, rs79272399, rs71313929, rs12211900, rs71379941, rs75217739, rs139788123, rs79986886, rs116448748, rs77911328, rs145225093, rs76211390, rs117241924, rs200676466, rs4972833, rs148123087, rs181841887) and early onset grade 3+toxicity of any type. Results when these 26 SNPs were included were AUC 72.8% (95% CI: 68.1-77.6%), specificity of 69.50% and sensitivity of 64.79%, at a threshold of −0.005 for early onset grade 3+toxicity and AUC 63.9 (95% CI 60.0-67.80%), specificity 75.8% sensitivity 47.73% at a threshold of 0.004 for any cycle grade 3+toxicity of any type.

The polymorphisms identified in this analysis are believed to represent a robust classifier for capecitabine-induced toxicity. The analysis described herein also suggests that these polymorphisms may be useful in predicting toxicity in other 5-FU monotherapy regimens.

TABLE 4
Top independent SNPs from GWAS of early onset and all cycles capecitabine toxicity from conditional
and joint (‘COJO’) analysis using the expected genotype dosage method to handle genotype data.

| | | | | | Tox | | | | |
|---|---|---|---|---|---|---|---|---|---|
| Early/All | | | | Toxicity | associated | | Lower | Upper | |
| cycles | Toxicity | SNP | rsIDs | risk allele | allele freq | OR | CI | CI | P value |
| Early | Diarrhoea | 16: 73570551_T_C | rs8048167 | C | 0.184 | 3.699 | 2.278 | 6.007 | 1.24E−07 |
| Early | Diarrhoea | 21: 21616413_C_CT | rs10654492 | del T | 0.323 | 3.448 | 5.495 | 2.16 | 2.00E−07 |
| Early | Diarrhoea | 18: 57863787_A_G | rs35087079 | G | 0.016 | 13.205 | 4.974 | 35.061 | 2.22E−07 |
| Early | Diarrhoea | 8: 26875681_C_A | rs117686094 | A | 0.033 | 5.482 | 2.872 | 10.465 | 2.50E−07 |
| Early | Diarrhoea | 7: 31923305_C_T | rs117260063 | T | 0.036 | 6.567 | 3.21 | 13.439 | 2.58E−07 |
| Early | Diarrhoea | 12: 124074365_C_T | rs12231444 | T | 0.034 | 5.897 | 2.989 | 11.634 | 3.09E−07 |
| Early | Diarrhoea | 3: 61476710_TA_T | rs1491207661 | ins A | 0.152 | 3.44 | 5.56 | 2.13 | 4.76E−07 |
| Early | Diarrhoea | 16: 85981482_C_T | rs11643168 | T | 0.069 | 4.803 | 2.607 | 8.848 | 4.82E−07 |
| All | Diarrhoea | 6: 37510161_T_C | rs12211900 | C | 0.106 | 2.875 | 1.973 | 4.191 | 3.92E−08 |
| All | Diarrhoea | 17: 63589380_T_C | rs71379941 | C | 0.02 | 8.657 | 3.754 | 19.964 | 4.14E−07 |
| Early | Global | 22: 19713353_G_C | rs71313929 | G | 0.614 | 2.667 | 3.906 | 1.828 | 3.74E−07 |
| All | Haem | 22: 45577373_C_T | rs75217739 | T | 0.059 | 30.074 | 8.243 | 109.726 | 2.55E−07 |
| All | Haem | 12: 18946760_G_A | rs139788123 | A | 0.032 | 21.365 | 6.593 | 69.229 | 3.32E−07 |
| Early | Hindfoot | 7: 71166314_G_A | rs181841887 | A | 0.025 | 6.409 | 3.33 | 12.334 | 2.68E−08 |
| All | Hindfoot | 5: 165441781_A_G | rs79986886 | G | 0.042 | 4.119 | 2.445 | 6.938 | 1.03E−07 |
| All | Hindfoot | 1: 97490443_G_A | rs116448748 | A | 0.029 | 4.566 | 2.554 | 8.162 | 3.00E−07 |
| Early | Mucositis | 14: 45617764_T_C | rs76211390 | C | 0.038 | 37.136 | 9.792 | 140.84 | 1.07E−07 |
| Early | Mucositis | 14: 99945412_A_C | rs79272399 | C | 0.019 | 41.555 | 9.875 | 174.876 | 3.71E−07 |
| All | Mucositis | 2: 208627843_C_T | rs77911328 | T | 0.019 | 45.28 | 11.195 | 183.144 | 8.90E−08 |
| All | Mucositis | 9: 84407550_T_C | rs145225093 | C | 0.016 | 23.807 | 7.16 | 79.156 | 2.32E−07 |
| All | Mucositis | 14: 45617764_T_C | rs76211390 | C | 0.038 | 19.309 | 6.205 | 60.088 | 3.20E−07 |
| All | Mucositis | 8: 137378694_C_A | rs117241924 | A | 0.015 | 42.871 | 10.069 | 182.526 | 3.68E−07 |
| Early | Vomiting | 11: 97684338_C_T | rs193272564 | T | 0.013 | 70.806 | 14.837 | 337.906 | 9.17E−08 |
| Early | Vomiting | 13: 70848486_AAAC_A | rs140744481 | A | 0.016 | 51.546 | 11.167 | 237.929 | 4.37E−07 |
| All | Vomiting | 11: 121702545_C_CT | rs200676466 | CT | 0.018 | 39.344 | 10.47 | 147.843 | 5.41E−08 |
| All | Vomiting | 2: 173372371_G_A | rs4972833 | A | 0.024 | 25.395 | 7.54 | 85.534 | 1.78E−07 |
| All | Vomiting | 2: 213644924_T_C | rs148123087 | C | 0.021 | 22.763 | 6.891 | 75.191 | 2.96E−07 |

“Chr” = chromosome; “Freq” = frequency; “OR” = odds ratio; “Lower CI” = lower confidence interval; “Upper CI” = upper confidence interval. “Toxicity risk allele” = allele associated with toxicity risk (e.g. for rs12211900, 6: 37510161_C is associated with toxicity risk).

TABLE 5
Gene annotations for the toxicity associated alleles
identified using the expected genotype dosages.

| rsIDs | Consequences | Mapped gene |
|---|---|---|
| rs8048167 | intergenic | LINC01568(dist = 115256), LOC101928035(dist = 655740) |
| rs10654492 | intergenic | LINC01683(dist = 344344), LINC02573(dist = 12762) |
| rs35087079 | intergenic | PMAIP1(dist = 292249), MC4R(dist = 174777) |
| rs117686094 | intergenic | ADRA1A(dist = 150921), STMN4(dist = 217159) |
| rs117260063 | intronic | PDE1C |
| rs12231444 | intronic | TMED2 |
| rs1491207661 | intergenic | FHIT(dist = 239575), PTPRG(dist = 70532) |
| rs11643168 | intergenic | IRF8(dist = 25267), LINC01082(dist = 248305) |
| rs12211900 | intergenic | LINC02520(dist = 6105), MIR4462(dist = 12980) |
| rs71379941 | intergenic | AXIN2(dist = 31614), CEP112(dist = 42278) |
| rs71313929 | intergenic | SEPT5-GP1BB(dist = 1056), TBX1(dist = 30873) |
| rs75217739 | ncRNA_intronic | LOC105373064 |
| rs139788123 | intergenic | CAPZA3(dist = 54638), PLEKHA5(dist = 335866) |
| rs181841887 | intronic | GALNT17 |
| rs79986886 | intergenic | LOC102546299(dist = 1471792), LINC01947(dist = 890446) |
| rs116448748 | intergenic | PTBP2(dist = 209838), DPYD(dist = 52857) |
| rs76211390 | intronic | FANCM |
| rs79272399 | intronic | SETD3 |
| rs77911328 | UTR3 | FZD5(NM_003468:c. *3863G > A) |
| rs145225093 | intergenic | LOC101927502(dist = 15736), SPATA31D5P(dist = 120802) |
| rs117241924 | intergenic | KHDRBS3(dist = 718842), LINC02055(dist = 164347) |
| rs193272564 | intergenic | LOC105369443(dist = 1436424), CNTN5(dist = 1207368) |
| rs140744481 | intergenic | ATXN8OS(dist = 134601), LINC00348(dist = 740784) |
| rs200676466 | intergenic | SORL1(dist = 198074), MIR100HG(dist = 196492) |
| rs4972833 | intergenic | ITGA6(dist = 1188), PDK1(dist = 48326) |
| rs148123087 | intergenic | ERBB4(dist = 241572), LINC01878(dist = 15131) |

1. A method of screening for risk of capecitabine-induced toxicity in a subject, comprising screening the subject for the presence of at least one polymorphism selected from: rs12211900; rs8048167; rs10654492; rs35087079; rs117686094; rs117260063; rs12231444; rs1491207661; rs11643168; rs193272564; rs140744481; rs76211390; rs79272399; rs71313929; rs71379941; rs75217739; rs139788123; rs79986886; rs116448748; rs77911328; rs145225093; rs117241924; rs200676466; rs4972833; rs148123087; rs181841887; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836;
wherein:
i. the presence of said at least one polymorphism indicates an increased risk of developing capecitabine-induced toxicity compared to a subject which does not possess said at least one polymorphism; and
ii. a negative result indicates no increased risk or a decreased risk of developing capecitabine-induced toxicity compared to a subject which possesses said at least one polymorphism.

2. The method according to claim 1, comprising screening for the presence of at least one polymorphism selected from rs12211900; rs8048167; rs10654492; rs35087079; rs117686094; rs117260063; rs12231444; rs1491207661; rs11643168; rs193272564; rs140744481; rs76211390; rs79272399; rs71313929; rs71379941; rs75217739; rs139788123; rs79986886; rs116448748; rs77911328; rs145225093; rs117241924; rs200676466; rs4972833; rs48123087; and rs181841887.

3. The method according to claim 1, comprising screening for the presence of at least one polymorphism selected from rs76211390 and rs79272399.

4. The method according to claim 1, comprising screening for the presence of at least one polymorphism selected from rs76211390; rs77911328; rs145225093; and rs117241924.

5. The method according to claim 1, comprising screening for the presence of at least one polymorphism selected from rs76211390; rs79272399; rs77911328; rs145225093; and rs117241924.

6. The method according to claim 1, comprising screening for the presence of at least one polymorphism selected from rs75217739 and rs139788123.

7. The method according to claim 1, comprising screening for the presence of at least one polymorphism selected from rs8048167; rs10654492; rs35087079; rs117686094; rs117260063; rs12231444; rs1491207661; and rs11643168.

8. The method according to claim 1, comprising screening for the presence of at least one polymorphism selected from rs12211900 and rs71379941.

9. The method according to claim 1, comprising screening for the presence of at least one polymorphism selected from rs8048167; rs10654492; rs35087079; rs117686094; rs117260063; rs12231444; rs1491207661; rs11643168; rs12211900; and rs71379941.

10. The method according to claim 1, comprising screening for the presence of at least one polymorphism selected from rs193272564 and rs140744481.

11. The method according to claim 1, comprising screening for the presence of at least one polymorphism selected from rs200676466; rs4972833; and rs148123087.

12. The method according to claim 1, comprising screening for the presence of at least one polymorphism selected from rs193272564; rs140744481; rs200676466; rs4972833; and rs148123087

13. The method according to claim 1, comprising screening for the presence rs181841887.

14. The method according to claim 1, comprising screening for the presence of at least one polymorphism selected from rs79986886 and rs116448748.

15. The method according to claim 1, comprising screening for the presence of at least one polymorphism selected from rs181841887; rs79986886; and rs116448748.

16. The method according to claim 1, comprising screening for the presence of at least one polymorphism selected from rs8048167; rs10654492; rs35087079; rs117686094; rs117260063; rs12231444; rs1491207661; rs11643168; rs71313929; rs181841887; rs76211390; rs79272399; rs193272564; and rs140744481.

17. The method according to claim 1, comprising screening for the presence of at least one polymorphism selected from rs12211900; rs71379941; rs75217739; rs139788123; rs79986886; rs116448748; rs77911328; rs145225093; rs76211390; rs117241924; rs200676466; rs4972833; and rs148123087.

18. The method according to claim 1, comprising screening for the presence of at least one polymorphism selected from rs12211900; rs698607; rs115143613; rs147827021; rs199983532; rs1327306; rs4792825; rs78361592; rs6137844; rs2929329; rs35741121; rs56329496; rs117810108; rs149994224; rs11024884; rs111378975; rs2420201; rs11375020; rs75504333; rs11447080; rs74380254; rs66842063; rs180742844; rs527945 and rs73039836.

19. The method according to claim 1, comprising screening for the presence of at least one polymorphism selected from rs147827021; rs149994224; and rs111378975

20. The method according to claim 1, comprising screening for the presence of at least one polymorphism selected from rs2929329; rs35741121; rs56329496; rs117810108; rs149994224; rs11024884; and rs111378975.

21. The method according to claim 1, comprising screening for the presence of at least one polymorphism selected from rs2420201; rs11375020; rs75504333; rs11447080; rs74380254; rs66842063; rs149994224; rs111378975; rs180742844; and rs527945.

22. The method according to claim 1, comprising screening for the presence of at least one polymorphism selected from rs2929329; rs35741121; rs56329496; rs117810108; rs149994224; rs11024884; rs111378975, rs2420201; rs11375020; rs75504333; rs11447080; rs74380254; rs66842063; rs180742844; and rs527945.

23. The method according to claim 1, comprising screening for the presence of at least one polymorphism selected from rs698607; rs115143613; rs147827021; rs199983532; rs1327306; rs4792825; and rs78361592.

24. The method according to claim 1, comprising screening for the presence of at least one polymorphism selected from rs147827021 and rs6137844.

25. The method according to claim 1, comprising screening for the presence of at least one polymorphism selected from rs698607; rs115143613; rs147827021; rs199983532; rs1327306; rs4792825; rs78361592; and rs6137844.

26. The method according to claim 1, comprising screening for the presence of at least one polymorphism selected from rs698607; rs115143613; rs147827021; rs199983532; rs1327306; rs4792825; rs78361592; rs2929329; rs35741121; rs56329496; rs117810108; rs149994224; rs11024884; and rs111378975.

27. The method according to claim 1, comprising screening for the presence of at least one polymorphism selected from rs12211900; rs147827021; rs6137844; rs2420201; rs11375020; rs75504333; rs11447080; rs74380254; rs66842063; rs149994224; rs111378975; rs180742844; rs527945; and rs73039836.

28. The method according to claim 1, comprising screening for the presence of at least one polymorphism selected from rs698607; rs115143613; rs147827021; rs199983532; rs1327306; rs4792825; rs78361592; rs2929329; rs35741121; rs56329496; rs117810108; rs149994224; rs11024884; rs111378975; rs12211900; rs6137844; rs2420201; rs11375020; rs75504333; rs11447080; rs74380254; rs66842063; rs180742844; rs527945; and rs73039836.

29. The method according to claim 1, wherein the subject is a cancer patient.

30. The method according to claim 29, wherein the patient has a solid tumour cancer.

31. The method according to claim 30, wherein the patient has colorectal cancer (CRC), or breast cancer, or gastrointestinal cancer.

32. The method according to claim 1, wherein the subject is undergoing chemotherapy with 5-FU.

33. The method according to claim 32, wherein the subject is undergoing capecitabine monotherapy.

34. The method according to claim 1, wherein the screening is carried out on a fluid sample from a patient.

35. The method according to claim 34, wherein the fluid sample is a saliva, blood serum or plasma sample.

36. The method according to claim 1, wherein the screening is carried out on a solid sample from a patient.

37. The method according to claim 36, wherein the solid sample is from a biopsy.

38. The method according to claim 1, further comprising the step of monitoring the subject for one or more symptoms of capecitabine-induced toxicity in the event of a positive result.

39. The method according to claim 38, wherein the one or more symptoms include mucositis/stomatitis, haematological toxicity, diarrhoea, nausea and vomiting, myelosuppression, neutropaenia, thrombocytopaenia, and/or hand-foot syndrome (HFS).

40. The method according to claim 39, wherein the one or more symptoms include mucositis/stomatitis and/or haematological toxicity.

41. The method of claim 1, wherein screening is carried out through logistic regression.

42. The method according to claim 41, wherein a genetic score ΣβiNi is assigned to each individual, where βi is the beta coefficient of the ith SNP significantly associated with global toxicity in a logistic regression model, and Ni is the number of harmful alleles carried by that individual at that locus.

43. The method according to claim 1, wherein the capecitabine-induced toxicity is early onset capecitabine-induced toxicity.
